(19) United States
(12) Patent Application Publication
(10) Pub. No.: US 2024/0159850 A1
(43) Pub. Date: May 16, 2024

(54) METHOD AND SYSTEM FOR SYNTHESIZING MAGNETIC RESONANCE IMAGES

(71) Applicant: Ramot at Tel-Aviv University Ltd., Tel-Aviv (IL)

(72) Inventors: Noam BEN-ELIEZER, Tel-Aviv (IL); Chen SOLOMON, Tel-Aviv (IL)

(73) Assignee: Ramot at Tel-Aviv University Ltd., Tel-Aviv (IL)

(21) Appl. No.: 18/403,871

(22) Filed: Jan. 4, 2024

Related U.S. Application Data
(63) Continuation of application No. PCT/IL2022/050721, filed on Jul. 5, 2022.
(60) Provisional application No. 63/218,410, filed on Jul. 5, 2021.
(60) Provisional application No. 63/218,414, filed on Jul. 5, 2021.

(51) Int. Cl. G01R 33/56; G01R 33/54; G06N 3/08; G06T 11/00

(52) U.S. Cl. CPC G01R 33/5608; G01R 33/546; G06N 3/08; G06T 11/00; G06T 2200/24; G06T 2210/41

(57) ABSTRACT

A method of synthesizing a magnetic resonance (MR) image, comprises obtaining a quantitative MRI (qMRI) map of values of an MRI parameter, modulating values of the MRI parameter within a region of the qMRI map to mimic a tissue pathology therein, and generating an MR image based on the modulated qMRI map.

### RELATED APPLICATIONS

This application is a Continuation of PCT Patent Application No. PCT/IL2022/050721 having International filing date of Jul. 5, 2022, which claims the benefit of priority under 35 USC § 119(e) of U.S. Provisional Patent Application Nos. 63/218,410 and 63/218,414, both filed on Jul. 5, 2021. The contents of the above applications are all incorporated by reference as if fully set forth herein in their entirety.

### FIELD AND BACKGROUND OF THE INVENTION

The present invention, in some embodiments thereof, relates to magnetic resonance imaging and, more particularly, but not exclusively, to a method and system for synthesizing magnetic resonance images.

Magnetic Resonance Imaging (MRI) is a method to obtain an image representing the chemical and physical microscopic properties of materials, by utilizing a quantum mechanical phenomenon, named Nuclear Magnetic Resonance (NMR), in which a system of spins, placed in a magnetic field resonantly absorb energy, when applied with a certain frequency.

A nucleus can experience NMR only if it has a nonzero nuclear spin ‘I’, i.e., the nucleus has at least one unpaired nucleon. Examples of non-zero spin nuclei frequently used in MRI include ¹H (I=1/2), ²H (I=1), ²³Na (I=3/2), etc. When placed in a magnetic field, a nucleus having a spin I is allowed to be in a discrete set of energy levels, the number of which is determined by I, and the separation of which is determined by the gyromagnetic ratio of the nucleus and by the magnetic field. Under the influence of a small perturbation, manifested as a radiofrequency magnetic field, which rotates about the direction of a primary static magnetic field, the nucleus has a time dependent probability to experience a transition from one energy level to another. With a specific frequency of the rotating magnetic field, the transition probability may reach the value of unity. Hence at certain times, a transition is forced on the nucleus, even though the rotating magnetic field may be of small magnitude relative to the primary magnetic field. For an ensemble of spin I nuclei the transitions are realized through a change in the overall magnetization.

Once a change in the magnetization occurs, a system of spins tends to restore its magnetization to a longitudinal equilibrium value, by the thermodynamic principle of minimal energy. The time constant which control the elapsed time for the system to return to the equilibrium value is called “spin-lattice relaxation time” or “longitudinal relaxation time” and is denoted as T₁. An additional time constant, T₂ (≤T1), called “spin-spin relaxation time” or “transverse relaxation time”, controls the elapsed time in which the transverse magnetization diminishes, by the principle of maximal entropy.

In MRI, a static magnetic field having a gradient is applied on an object, thereby creating, at each region of the object, a unique magnetic field. By detecting the NMR signal, and by knowing the prescribed magnetic field gradient, the position of each region of the object can be deduced, thereby creating an image of the object. In many MRI techniques, differences between characteristic T₂ values are used to create visually qualitative contrast in the magnetic resonance image. It is however recognized that further information can be obtained by quantitative characterization of the T₂ relaxation time constant at each region of the substance under investigation.

Quantitative MRI (qMRI) is a technique that maps one or more of the aforementioned relaxation times or other parameters obtainable by MRI over some region from which the magnetic resonance (MR) signal is acquired. Such mapping often makes use of analytical models of MR signals to model the tissue, and provide information specific to pathologies or other tissue features. For example, qMRI has been shown to be able to identify pathologies in white matter tissue of multiple sclerotic patients, which look completely normal when examined using conventional MRI techniques [Shepherd, et al. Neurolmage Clin. 14, 363-370 (2017), Gracien, et al., J. Magn. Reson. Imaging 44, 1600-1607 (2016), Hagiwara et al., Am. J. Neuroradiol. 38, 237-242 (2017)].

### SUMMARY OF THE INVENTION

According to an aspect of some embodiments of the present invention there is provided a method of synthesizing a magnetic resonance (MR) image. The method comprises: obtaining a quantitative MRI (qMRI) map of values of an MRI parameter; modulating values of the MRI parameter within a region of the qMRI map, to mimic a tissue pathology therein, thereby providing a modulated qMRI map; and generating an MR image based on the modulated qMRI map, thereby synthesizing the MR image.

According to some embodiments of the invention the invention the method comprises generating the qMRI map.

According to some embodiments of the invention the qMRI map is generated based on an MR signal acquired from a subject.

According to some embodiments of the invention the subject is a healthy subject.

According to some embodiments of the invention the invention the method comprises accessing a computer readable medium storing a database having a plurality of entries each associating a database pathology to a database value or range of values of at least one MRI parameter, and searching the database for an entry having a database pathology matching the tissue pathology, wherein the modulation of the values of the parameter is based on a database value or range of values of the found entry.

According to some embodiments of the invention the invention the method comprises randomly selecting the region.

According to some embodiments of the invention the modulation is along a randomly selected pattern within the region.

According to some embodiments of the invention the region is predetermined.

According to some embodiments of the invention the invention the method comprises accessing a computer readable medium storing a database having a plurality of entries each associating a database pathology to a database morphology, and searching the database for an entry having a database pathology matching the tissue pathology, wherein the modulation of the values within the region is along a pattern selected based on a database morphology of the found entry.

According to some embodiments of the invention the invention the method comprises applying at least one morphology varying operation to the database morphology of the found entry.

According to some embodiments of the invention at least one of a type and an extent of the morphology varying operation is selected randomly.

According to some embodiments of the invention the database morphology corresponds to an orientation of muscle fibers.

According to some embodiments of the invention the database morphology corresponds to an orientation of neural fibers.

According to some embodiments of the invention the method comprises receiving input pertaining to a severity level of the tissue pathology, wherein the modulation of the values is based on the received severity level.

According to some embodiments of the invention the method comprises generating a simultaneous graphical output of the synthesized the MR image, and an MR image corresponding to the qMRI map prior to the modulation.

According to an aspect of some embodiments of the present invention there is provided a computer software product, comprising a computer-readable medium in which program instructions are stored, which instructions, when read by a data processor, cause the data processor to receive a qMRI map of values of at least one MRI parameter and execute the method as delineated above and optionally and preferably as further detailed below.

According to an aspect of some embodiments of the present invention there is provided a method of training an artificial neural network. The method comprises executing the method as delineated above and optionally and preferably as further detailed below a plurality of times to respectively synthesize a plurality of MR images, each associated with at least one tissue pathology. The method also comprises feeding the artificial neural network with the synthesized MR images and the respective tissue pathologies, to obtain weight parameters for the artificial neural network, and storing the weight parameters in a computer readable medium.

According to some embodiments of the invention the method comprises re-executing the synthesis an additional plurality of times to respectively synthesize an additional plurality of MR images, each associated with at least one tissue pathology, validating the weight parameters by feeding the artificial neural network with each of the additional plurality of synthesized MR images, and comparing an output of the artificial neural network with a respective tissue pathology. A report indicative of the validation can be generated based on the comparison.

According to an aspect of some embodiments of the present invention there is provided a computer software product, comprising a computer-readable medium in which program instructions are stored, which instructions, when read by a data processor, cause the data processor to receive a qMRI map of values of at least one MRI parameter and execute the method as delineated above and optionally and preferably as further detailed below.

According to an aspect of some embodiments of the present invention there is provided a computer software product for training a user. The computer software product comprises a computer-readable medium in which program instructions are stored, which instructions, when read by a data processor, cause the data processor to: display on a display device a graphical user interface (GUI) having a training activation control; automatically execute the synthesis method as delineated above and optionally and preferably as further detailed below, responsively to an activation of the control by the user, and generate a graphical output of the synthesized the MR image on the GUI.

According to some embodiments of the invention the program instructions, when read by a data processor, cause the data processor to synthesize an ordered set of MR images mimicking the tissue pathology, and to generate a graphical output separately for each of the MR images on the GUI.

According to some embodiments of the invention the set of MR images comprises synthesized MR images at which a visibility of the synthesized pathology gradually increases or decreases.

According to some embodiments of the invention the set of MR images comprises synthesized MR images at which a severity level of the synthesized pathology gradually increases or decreases.

According to some embodiments of the invention the set of MR images comprises synthesized MR images at which a size of the region gradually increases or decreases.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram describing a method suitable for synthesizing a magnetic resonance image according to various exemplary embodiments of the present invention.

FIG. 2 is a schematic illustration of a representative example of a database suitable for use with the method according to some embodiments of the present invention.

FIG. 3 is a flowchart diagram describing a method suitable for training an artificial neural network, according to some embodiments of the present invention.

FIG. 4 is a schematic illustration of a computing platform suitable for execution selected operations of the method according to some embodiments of the present invention.

FIG. 5 is a schematic illustration of a representative example of a graphical user interface suitable for training a user to inspect and analyze MR images, according to some embodiments of the present invention.

FIGS. 6A-D show demonstration of trial images formation, obtained during experiments performed according to some embodiments of the present invention.

FIGS. 7A-C show AUC detection rate as a function lesion severity, as obtained during experiments performed according to some embodiments of the present invention.

FIGS. 8A-C relate demyelination processes, their effect on the T₂ relaxation time and appearance in T₂-weighted FLAIR images. FIG. 8A illustrates that inflammation leads to demyelination of axons. FIG. 8B shows inflamed areas that are characterized with elevated qT₂.

FIG. 8C shows regions of elevated qT₂ that appear as hyperintensities in FLAIR images. Lesions are emphasized by white bounding boxes.

FIG. 9 shows a series of operations for synthesizing MRI used in experiments performed according to some embodiments of the present invention. In operation A T1 w image is acquired from a healthy subject. In operation B, T2w scans of various TEs from are acquired from a healthy subject. In operation C, WM segmentation is generated using Freesurfer software. In operation D, T2 map is generated using the EMC algorithm. In operation E PD map is generated using the EMC algorithm. In operation F randomization is applied to a convex ROI in the WM, whose values dictate pathological T2 changes. In operation G a lesioned T2 map is generated by voxel-wise multiplication of the ROI and the T2 map. In operation H, a T2-FLAIR image is synthesized using an analytical signal model.

FIGS. 10A-D show synthetic lesion embedded on a 2D FLAIR image in experiments performed according to some embodiments of the present invention, at three different severity levels. FIG. 10A shows a WM region which was randomly chosen for the synthetic MS lesion. FIGS. 10B, 10C and 10D show a synthesized lesion obtained by changing the underlying values of the tissue's T2 relaxation times, by 30%, 18%, and 6%, respectively.

FIGS. 11A-C show a scheme of a psychophysical trial performed according to some embodiments of the present invention. FIG. 11A illustrates a training phase, wherein pairs of images in which two out of three right-hand images are lesioned, and in which the left-hand images exhibit the same slice with no simulated lesion were presented to a radiologist. Lesions were highlighted by the radiologist, when found. FIG. 11B illustrates the test phase, wherein one image was present at a time. At this phase, two out of three images were lesioned in various severity levels. FIG. 11C shows raw data illustration in a confusion matrix, showing correct classifications (TP—true positive/hit, TN—true negative/correct rejection) and wrong classifications (FP—false positive/false alarm, FN—false negative/miss).

FIGS. 12A-B are schematic illustrations of a convolutional neural network used in experiments performed according to some embodiments of the present invention.

FIG. 13 shows TP and FP rates for radiologic and CAD as a function of the lesion severity. TP rate for CAD was significantly higher than that of radiologists (p-value<0.05) at middle-low severity levels of 9-15% elevation in T2, and comparable at higher and lower levels. FP rate (dashed line) for CAD is significantly lower (p-value<0.001) than that of radiologists.

FIG. 14 shows diagnostic odd ratios for radiologists and CAD as a function of the lesion severity. Error bars indicate 95% confidence intervals (CIs). ORs for both techniques increase with lesion severity. ORs for CAD are significantly higher than ORs for radiologists in the four lowest severity levels (≤15% elevation in T2 relaxation times) and are comparable for higher lesion severity. Statistically significant difference: p<0.0001 with Z-test for log(OR).

FIG. 15 is a schematic illustration of a process used for converting quantitative maps to FLAIR contrast. In operation A, proton density weighted is generated using the EMC algorithm. In operation B, qT₂ map is generated using the EMC algorithm. In operation C, qT₁ map is linearly estimated from qT₂ map. In operation D, different FLAIR contrasts represent the signal during each of the turbo spin-echoes. In operation E, k-spaces of the turbo spin-echoes are computed using DFT. In operation F, readout lines are sampled at different echoes form the k-space for the resulting FLAIR image. In operation G, FLAIR image is computed from the k-space.

FIGS. 16A and 16B show an axial slice of a brain acquired using a FLAIR protocol (FIG. 16A), and a synthetic FLAIR image generated from a qT₂ map of the same slice (FIG. 16B).

### DESCRIPTION OF SPECIFIC EMBODIMENTS OF THE INVENTION

The present invention, in some embodiments thereof, relates to magnetic resonance imaging and, more particularly, but not exclusively, to a method and system for synthesizing magnetic resonance images.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

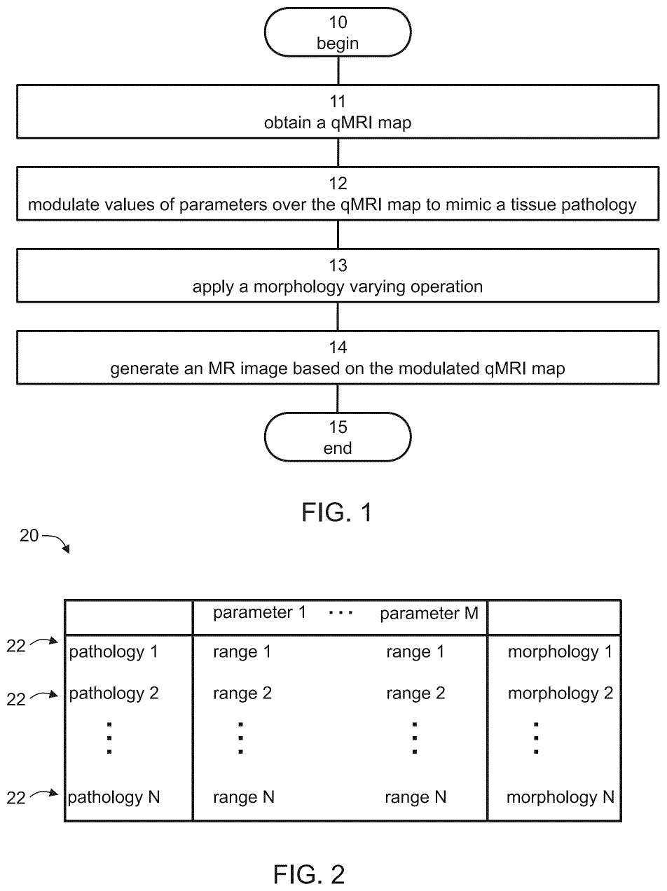

FIG. 1 is a flowchart diagram of a method suitable for synthesizing a magnetic resonance (MR) image according to various exemplary embodiments of the present invention. It is to be understood that, unless otherwise defined, the operations described herein below can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be implemented by a data processing system, e.g., a dedicated circuitry or a general purpose computer, configured for receiving MR data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

The method begins at 10 and optionally and preferably continues to 11 at which a quantitative MRI (qMRI) map of values of one or more MRI parameters is obtained.

The MRI parameter can any parameter that is obtainable from an MR signal. Typically, the MRI parameter is a relaxation times, e.g., T₁, T₂, T₂*, but it can alternatively include a diffusion coefficient, magnetization transfer, proton density, or the like.

The qMRI map is a data structure that describes the distribution of values of the respective MRI parameter, by associating each value with a point defined over a system of coordinates, and optionally and preferably also with a neighborhood of that point. The qMRI map can be a two-dimensional map in which case the system of coordinates is a two-dimensional system of coordinates, or a three-dimensional map in which case the system of coordinates is a three-dimensional system of coordinates. The system of coordinate is typically Cartesian, but a curvilinear system of coordinates is also contemplated.

In some embodiments of the present invention the qMRI map includes a plurality of two-dimensional sub-maps arranged in a stack, such that each sub-map describes the distribution the distribution of values of the respective MRI parameter over a two-dimensional slice (e.g., a planar slice) of a three-dimensional space. Each slice can represent, for example, a different depth from which the MR signal is obtained.

When the qMRI map includes values of more than one type of MRI parameter (e.g., includes both T₂ and T₂*values or the like), one or more points over the system of coordinates are associated with more than one value, a value for each type of MRI parameter. Equivalently, the qMRI map can be a collection of single-parameter qMRI sub-maps, wherein each such single-parameter qMRI sub-map associates values of one type of MRI parameter with points over the system of coordinates, optionally together their respective neighborhoods.

The qMRI map can be obtained from an external source (e.g., read from a computer readable medium), or generated by the method of the present embodiments. The qMRI map is typically generated from MR data describing an MR signal acquired by an MRI scanner from an organ of a subject. This can be done using any technique known in the art, such as, but not limited to, one of the techniques disclosed in the textbook: Quantitative MRI of the Brain: Principles of Physical Measurement, Second edition, Edited By Mara Cercignani, Nicholas G. Dowell, Paul S. Tofts, 2018, particularly but not exclusively chapters 4, 7, 10, 11, and 12 therein. A specific example of a technique suitable for generating the qMRI map from the MR data, particularly for the case in which the MRI parameter is T₂, is described in Ben-eliezer et al., Magn Reson Med 2015; 73(2):809-817. When the MRI parameter is T₂, the qMRI map can be generated from the MR data using the technique described in Marques et al., Marques et al., Neuroimage 2010 49(2):1271-81, when the MRI parameter is diffusion coefficient, the qMRI map can be generated from the MR data using the technique described in Basser et al., Biophysical Journal 1994 66:259-267, and when the MRI parameter is T₂*, the qMRI map can be generated from the MR data using the technique described in Haacke et al., 2015, Magnetic Resonance Imaging, Volume 33, Issue 1, pages 1-25.

Preferably, but not necessarily, the subject from which the MR signal is acquired is a healthy subject. This is advantages in particular in cases in which it is desired to synthesize MR images that have a particular pathology or collection of pathologies, in which case using MR data obtained from a healthy subject ensures than no undesired pathologies are manifested in the synthesized images. Alternatively, the subject from which the MR signal is acquired already has one or more pathology in the imaged region. This is advantages in particular in cases in which it is desired to synthesize MR images in a manner that the synthesized MR images mimic MR images depicting the same pathology or pathologies except that at MRI settings that are different from the settings used to acquire the MR signal. Thus, for example, suppose that an MR signal is acquired from a subject having a particular pathology using particular MRI settings (e.g., an MRI system of a particular type, employing a particular pulse sequence), and that this MR signal is processed to generate a qMRI map. As will be explained in greater detail below, the method of the present embodiments can use this qMRI map to synthesize an MR image that is similar to an MR image that would have been generated for the same subject with the same particular pathology, had the MR signal been acquired from using different MRI settings.

At 12 the method modulates values of one or more of the MRI parameters within a region of the qMRI map. The region preferably encompasses a portion of the map, but embodiments in which the region encompasses the entire map are also contemplated. The region forms a domain over the map which can be either a simply-connected or not simply-connected. Further, the region can be a continuous region, or it can be a collection of two or more disjoint sub-regions.

The location of the region with the qMRI map and/or its geometrical shape can be predetermined (for example, selected by a user before executing operation 12) or it can be selected by the method according to some criterion or set of criteria. For example, the method can randomly select the location and/or shape of the region upon each execution of the method. The method can also store in a computer readable medium a collection of region definitions, and select the region (e.g., randomly) from this collection. Also contemplated, are embodiments in which the method keeps, on computer readable medium, history records pertaining to previous execution of the method, and selects the region also based on these history records. For example, the method can use the history records to ensure that different regions (or similar regions, as the case may be) are selected for consecutive executions of the method.

The modulation is optionally and preferably selected so as to mimic a tissue pathology within the region. In some embodiments of the present invention, for at least one of the pathologies to be synthesized, the region within which the values of the MRI parameter(s) are modulated to mimic the pathology is smaller than a minimal size of a pathology of the same type that is distinguishable be a human observer. These embodiments are particularly useful in applications in which the synthesized pathology is used in training data and/or validation data for the training of a machine learning procedure. Small size pathology optionally and preferably occupies a region which is less than 1 mm or less than 0.5 mm or less than 0.1 mm along its largest diameter.

It is to be understood however, that in some embodiments of the present invention the region within which the values of the MRI parameter(s) are modulated to mimic the pathology is of larger size, e.g., more than 1 mm or more than 2 mm or more than 4 mm or more than 8 mm or more than 16 mm or more than 32 mm or more than 64 mm or more.

The modulation of the parameter(s) to mimic the tissue pathology within the region can be done in more than one way. In the simplest case, the method receives a range of values that is designated as a non-pathological or normal for the respective parameter (e.g., a range that would be typically obtainable from a healthy subject, given a typical MRI settings), and selects for that parameter at a particular point of the map a modified value that is outside this range. Such a selection is preferably repeated for a plurality of points within the region. Preferably, the method uses different ranges of values that is designated as a non-pathological or normal for two or more different points of the region.

Conveniently, the range to be used at a particular point can be selected based on the unmodified value of the parameter at that point, for example, by using the unmodified value as a pointer to the correct non-pathological range of values. For example, suppose that the MRI parameter is the spin-spin relaxation time T₂, and suppose that the unmodified value of T₂ at a particular point p₁ of the map is within some non-pathological range R₁ of values (e.g., a range of T₂ values that are characteristic to a healthy tissue of a particular type, such as, but not limited to, white matter, bone, etc.). In this case the method can modify the T₂ value at that particular point p₁ to a new value that is outside the non-pathological range R₁. When the unmodified value of T₂ at another point p₂≠p₁ is within another non-pathological range R₂≠R₁ of values (e.g., corresponding to a healthy tissue of a different type), the method can modify the T₂ value at this other point p₂ to a new value that is outside the other non-pathological range R₂, and so on.

In some embodiments of the present invention the modulation is selected to mimic a specific tissue pathology. In these embodiments the method obtains a specific tissue pathology and selects a modified value for the parameter that is characteristic to than pathology. For example, support that the MRI parameter is T₂, as before, and that the method obtains a pathology X. In this case the method can modify the T₂ value at that particular point p₁ to a new value that is within a range of values that is characteristic to pathology X for typical MRI settings. The method can obtain the specific tissue pathology by receiving it from external source (e.g., a user interface) or it can select the pathology from a list of pathologies, randomly or according to a predetermined criterion, e.g., based on history records of previously selected pathologies.

The present embodiments contemplate modulation of the parameter to mimic a one or more pathologies of brain tissue, bony tissue, muscle tissue, and/or blood vessel tissue. Representative examples of contemplated pathologies including, without limitation, multiple sclerosis, breast cancer, fatty liver, brain tumor, muscle dystrophy, prostate cancer, artery stenosis, mesenteric ischemia, granuloma, cyst, hemorrhage (intracerebral, extradural, or subdural), aneurysm, abscess, edema, adenopathy, pancreatic or other retroperitoneal lesion, stroke, dementia, hydrocephalus, brain trauma, distal myopathy, pericarditis, arthritis, arteriovenous malformation, bone infection, bone cancer, bone fractions, torn cartilage, ligament or tendon, hernia, spinal cord compression, pinched nerve,

Preferably, the method uses a database for selecting the values to be used for the modulation 12. The database can be stored on a computer readable medium and the method can access this database and use its entries for selecting the values for the modulation. A representative example of a database 20 suitable for the present embodiments is illustrated in FIG. 2.

Database 20 includes a plurality of entries 22 (N such entries are illustrated in FIG. 2). Each entry 22 associates a database pathology (shown in FIG. 2 as “pathology 1,” “pathology 2,” etc.) to a database value or range of values of one or more MRI parameter (M such parameters are illustrated in FIG. 2). N and M can be any positive integer, including 1. The method searches database 20 for an entry having a database pathology matching the obtained tissue pathology, and uses the database value or range of values of the found entry, for the modulation. For example, when the qMRI map includes values of a particular parameter, and the found entry includes a database range of values of that particular parameter for the obtained pathology, the method can modify the value of the parameter at a particular point of the map to be a value within the database range of values.

The method optionally and preferably selects among several databases such as database 20, wherein the selection is based on the type of tissue or organ that is to be described by the MR image to be synthesized. Thus, for example, the computer readable medium can include a first database like database 20 for synthesizing an MR image of a brain, a second database like database for synthesizing an MR image of lungs, a third database like database 20 for synthesizing an MR image of kidneys, a fourth database like database 20 for synthesizing an MR image of a limb, etc. The method can also selects among several databases such as database 20, wherein the selection is based on the MRI setting, such as, but not limited to, vendor, pulse sequence, and the like.

The method thus optionally and preferably receives input pertaining to the type of tissue and/or MRI setting, and selects the appropriate database, and search the database for an entry having a database pathology matching the obtained tissue pathology. It is appreciated, that database 20 can be constructed to include entries for different pathologies, different types of tissues, and different MRI settings, in which case a single database can be used.

The modified value of the MRI parameter that the method assigns to a particular point of the map during the modulation 12 is optionally and preferably selected by the method based on the severity level of the tissue pathology that is desired to be synthesized. Specifically, for higher severity level, the method selects a modified value that is farther from values or range of values which are identified as normal, and for lower severity level, the method selects a modified value that is closer to (yet different from) values or range of values which are identified as normal. When a database such as database 20 is employed, the method can selects for higher and lower severity levels modified values that are within sub-ranges of the range extracted from the database. Specifically, for higher severity level the method selects a modified value within a sub-range that is farther from values or range of values which are identified as normal, and for lower severity level, the method selects a modified value that within a sub-range that is closer to values or range of values which are identified as normal. The severity level can be predetermined, or, more preferably be received as a user input.

In some embodiments of the present invention the modified values of the MRI parameter(s) is/are only slightly different from the unmodified value(s), so as to mimics tissue pathology having a severity level which is on the border or below the minimal level that can be distinguished by a human observer. These embodiments are particularly useful in applications in which the synthesized pathology is used in training data and/or validation data for the training of a machine learning procedure. Low severity pathology can be synthesized by modifying the value of the MRI parameter to be within ±16% or ±14% or ±12% or ±10% or ±8% or ±6% or less of the unmodified value.

It is to be understood however, that in some embodiments of the present invention the severity of the pathology to be synthesized is higher, in which case the value of the MRI parameter is modified so that the difference between the modified and unmodified value, in absolute value, is at least 20% or at least 25% or at least 30% or at least 35% or at least 40% or at least 45% or at least 50% or more of the unmodified value.

The modulation 12, is preferably executed for each of a plurality of points forming a pattern over the region of the qMRI map. The pattern can be selected randomly or according to a criterion or a set of criteria. In some embodiments of the present invention the pattern is selected based on the neurobiological processes that underlie the appearance of the obtained pathology. For example, the pattern can be selected in advance to follow neural tractography. Such neural tractography can be obtained, e.g., by diffusion MRI, and stored in a computer readable medium. The method can access the computer readable medium and use the stored neural tractography for selecting the pattern.

In some embodiments of the present invention the pattern is selected base on a morphology that is stored in a database and is associated with the obtained pathology. For example, with reference to FIG. 2, the entries of database 20 (or a separate database, if desired), can include database morphologies (shown as “morphology 1,” “morphology 2,” etc.), which are respectively associated to the database pathologies 1, . . . N, so that a database pathology of a particular entry is associated to a specific database morphology. The database morphology can correspond, for example, to an orientation of muscle fibers, orientation of neural fibers, or the like.

The method can search the database 20 for an entry having a database pathology matching the obtained tissue pathology, and select the pattern based on the database morphology of the found entry.

In embodiments of the invention in which the pattern is not selected randomly or according to a selection criterion, the method optionally and preferably applies 13 one or more morphology varying operation to the pattern. For example, when the pattern is based on a database morphology extracted from a found entry of a database (e.g., database 20). Representative examples of morphology varying operations, suitable for the present embodiments, including, without limitation, erosion, dilation, mirroring, opening and closing. The morphology varying operation is preferably executed prior to the modulation 12, but the present embodiments also contemplate applying the operation after the values of the parameter(s) were modified. The type of morphology varying operation can be predetermined, provided as a user input, or selected by the method, e.g., randomly.

The method optionally and preferably proceeds to 14 at which an MR image is generated based on the modulated qMRI map. This can be done using any known technique for converting a qMRI map to an MR image. The generated MR image can be of any type known in the art. Representative Examples for such generated MR images include, without limitation, a T₁-weighted MR image, a T₂-weighted MR image, a diffusion weighted MR image, a T*₂-weighted MR image, a contrast enhanced T₁-weighted MR image, a fluid-attenuated inversion recovery (FLAIR) MR image, and the like.

In some embodiments of the present invention the method uses the qMRI map to estimate one or more MRI parameters of a type that is not mapped by the qMRI map. Such an estimation can be done by any technique known in the art. For example, the method can use a quantitative T₂ MRI map to estimate T₁ values, e.g., by calculating a linear regression of the correlation between T₂ and T₁ values in the respective organ based on published quantitative relaxation atlases. The estimated MRI parameter can be used to construct an additional qMRI map of the estimated parameter. In some embodiments of the present invention an MR image is generated based on the additional qMRI map. The generated MR image can be based only on the additional qMRI map, or, more preferably, on a combination of two or more qMRI maps (e.g., a combination of the modulated qMRI map with one or more additional qMRI maps constructed from estimated parameters).

Also contemplated, are embodiments in which a qMRI map of an unmodified parameter is combined with the modulated qMRI map, and optionally and preferably also one or more additional qMRI maps constructed from estimated parameters. The unmodified parameter is preferably of a different type than the modified parameter. For example, the modified parameter can be T₂ and the unmodified parameters can be proto density. The qMRI map of the unmodified parameter can be obtained from an external source (e.g., read from a computer readable medium), or generated by the method of the present embodiments. The qMRI map of the unmodified parameter is typically generated from MR data describing an MR signal acquired by an MRI scanner from the same organ. As a representative example, qMRI map of unmodified proton density values can be combined with a qMRI map of modified T₂ values and qMRI map of T₁ values that are estimated from the modified T₂ values, can be combined to generate a FLAIR MR image.

Techniques for converting qMRI maps into various types of MR images are known in the art and are found in, for example, www(dot)heartvista(dot)ai, www(dot)olea-medical(dot)com, and www(dot)radiopaedia(dot)org/articles/mri-sequences-overview.

The generated MR image can be transmitted to a remote location, stored in a computer readable medium, and/or displayed on a display device. In some embodiments of the present invention the method generates a simultaneous graphical output of the synthesized the MR image, and an MR image that corresponds to the qMRI map obtained at 11. The simultaneous display can be side-by-side, or in a superimposed manner, as desired.

The method ends at 15.

The method of the present embodiments can be used for various applications. In some embodiments of the present invention the method is used in training a machine learning procedure, such as, but not limited to, an artificial neural network.

Artificial neural networks are a class of algorithms based on a concept of inter-connected computer program objects referred to as neurons. In a typical artificial neural network, neurons contain data values, each of which affects the value of a connected neuron according to a pre-defined weight (also referred to as the “connection strength”), and whether the sum of connections to each particular neuron meets a pre-defined threshold. By determining proper connection strengths and threshold values (a process also referred to as training), an artificial neural network can achieve efficient recognition of image features. Oftentimes, these neurons are grouped into layers in order to make connections between groups more obvious and to each computation of values. Each layer of the network may have differing numbers of neurons, and these may or may not be related to particular qualities of the input data. An artificial neural network having a layered architecture belong to a class of machine learning procedure called “deep learning,” and is referred to as deep neural network (DNN).

In one implementation, called a fully-connected DNN, each of the neurons in a particular layer is connected to and provides input value to those in the next layer. These input values are then summed and this sum is compared to a bias, or threshold. If the value exceeds the threshold for a particular neuron, that neuron then holds a value which can be used as input to neurons in the next layer of neurons. This computation continues through the various layers of the neural network, until it reaches a final layer. At this point, the output of the DNN can be read from the values in the final layer.

Unlike fully-connected DNNs, convolutional neural networks (CNNs) operate by associating an array of values with each neuron, rather than a single value. The transformation of a neuron value for the subsequent layer is generalized from multiplication to convolution. When the neural network is a CNN, the training process adjusts convolutional kernels and bias matrices of the CNN so as to produce an output that resembles as much as possible known image features.

The final result of the training of an artificial neural network having a layered architecture (e.g., DNN, CNN) is a network having an input layer, at least one, more preferably a plurality of, hidden layers, and an output layer, with a learn value assigned to each component (neuron, layer, kernel, etc.) of the network. The trained network receives an MR image at its input layer and provides information pertaining to images feature present in the MR image at its output layer.

The training of an artificial neural network includes feeding the network with training data, for example data obtained from a cohort of subjects. The training data include MR images which are annotated by previously identified image features, such as regions exhibiting pathology and regions identified as healthy. Based on the MR images and the annotation information the network assigns values to each component of the network, thereby providing a trained network. Following the training, a validation process may optionally and preferably be applied to the artificial neural network, by feeding validation data to the network. The validation data is typically of similar type as the training data, except that only the MR images are fed to the trained network, without feeding the annotation information. The annotation information is used for validation by comparing the output of the trained network to the previously identified image features.

The Inventor appreciates that large amount of training and validating data requires extensive number of costly scans and time-consuming expert annotating processes. The Inventor found that the method of the present embodiments can realistically simulate the manifestation of disease on MR images and at different levels of severity, and can therefore be used to produces training and validating data at any amount while saving resources which would have otherwise allocated to annotating processes.

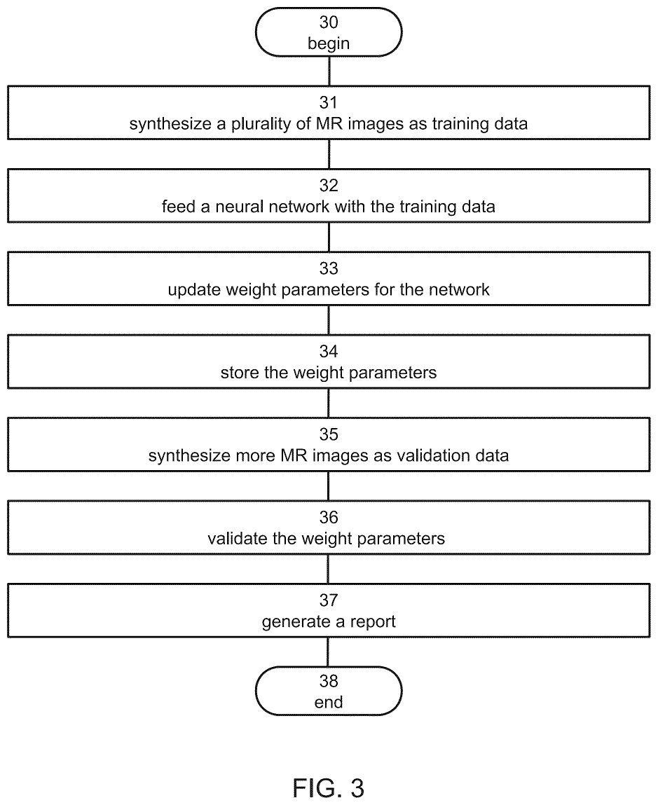

FIG. 3 is a flowchart diagram of a method suitable for training an artificial neural network, according to some embodiments of the present invention. The artificial neural network can be of any type, such as, but not limited to, a DNN or a CNN.

The method begins at 30 and continues to 31 at which a plurality of MR images are synthesized, to provide training data. Each synthesized MR image of the training data is associated with at least one tissue pathology. This is preferably done by executing the MR image synthesizing method described above with reference to FIGS. 1 and 2, a plurality of times. In some embodiments of the present invention at least a portion of the MR images of the training data, more preferably all the MR images of the training data, are synthesized from a single qMRI map, except for different pathologies and/or different sets of pathologies and/or different tissue types and/or different MRI settings as further detailed hereinabove. The qMRI map or maps used to synthesize the training data is/are optionally and preferably generated from an MR signal acquired from a healthy subject. Alternatively, the qMRI map or maps can be generated from an MR signal acquired from a subject having one or more known pathologies in organ from which the MR signal is acquired.

The method proceeds to 32 at which the artificial neural network is fed with the synthesized MR images and the respective tissue pathologies, and to 33 at which a weight parameter is updated for each component (neuron, layer, kernel, etc.) of the artificial neural network. The method proceeds to 34 at which the weight parameters are stored in a computer readable medium. In some embodiments of the present invention the method proceeds to 35 at which more MR images, each associated with one or more tissue pathology, are synthesized, to provide validating data. The validating data can be synthesized using the same technique employed to synthesized 31 the training data.

While operations 31 and 35 are described as two separate operations, it is to be understood that these operations can be combined. For example, the method can synthesize a plurality of MR images and then divide those images into a training set of MR images and a validation set of MR images.

The method proceeds to 36 at which the weight parameters are validated, by feeding the artificial neural network with the validating data, and comparing the output of the artificial neural network with the tissue pathology associated with each of the MR image of the validation data.

The method can then generate 37 a report indicative of the validation. The method ends at 38.

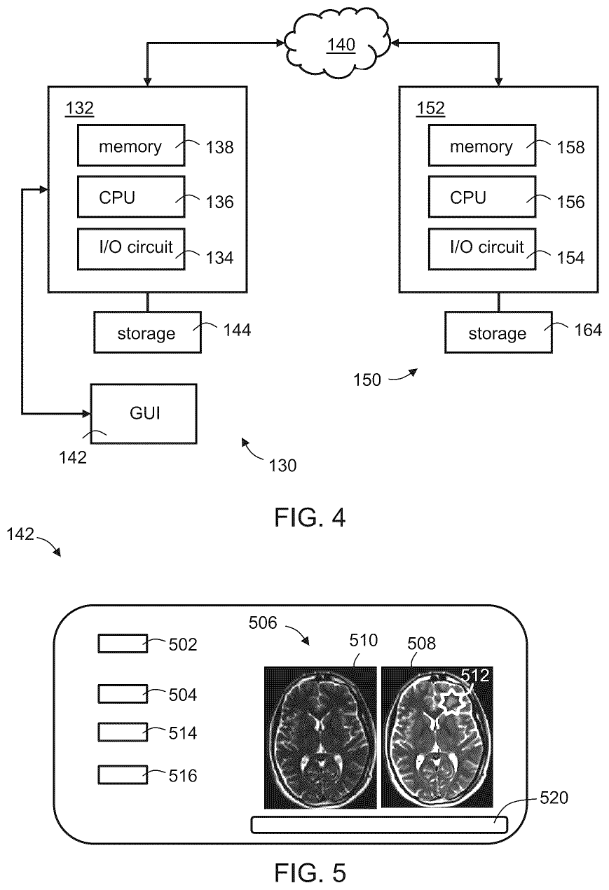

FIG. 4 is a schematic illustration of a client computer 130 having a hardware processor 132, which typically comprises an input/output (I/O) circuit 134, a hardware central processing unit (CPU) 136 (e.g., a hardware microprocessor), and a hardware memory 138 which typically includes both volatile memory and non-volatile memory. CPU 136 is in communication with I/O circuit 134 and memory 138. Client computer 130 preferably comprises a graphical user interface (GUI) 142 in communication with processor 132. I/O circuit 134 preferably communicates information in appropriately structured form to and from GUI 142. Also shown is a server computer 150 which can similarly include a hardware processor 152, an I/O circuit 154, a hardware CPU 156, a hardware memory 158. I/O circuits 134 and 154 of client 130 and server 150 computers can operate as transceivers that communicate information with each other via a wired or wireless communication. For example, client 130 and server 150 computers can communicate via a network 140, such as a local area network (LAN), a wide area network (WAN) or the Internet. Server computer 150 can be in some embodiments be a part of a cloud computing resource of a cloud computing facility in communication with client computer 130 over the network 140.

GUI 142 and processor 132 can be integrated together within the same housing or they can be separate units communicating with each other. GUI 142 can optionally and preferably be part of a system including a dedicated CPU and I/O circuits (not shown) to allow GUI 142 to communicate with processor 132. Processor 132 issues to GUI 142 graphical and textual output generated by CPU 136. Processor 132 also receives from GUI 142 signals pertaining to control commands generated by GUI 142 in response to user input. GUI 142 can be of any type known in the art, such as, but not limited to, a keyboard and a display, a touch screen, and the like.

Client 130 and server 150 computers can further comprise one or more computer-readable storage media 144, 164, respectively. Media 144 and 164 are preferably non-transitory storage media storing computer code instructions for executing the method as further detailed herein, and processors 132 and 152 execute these code instructions. The code instructions can be run by loading the respective code instructions into the respective execution memories 138 and 158 of the respective processors 132 and 152.

Each of storage media 144 and 164 can store program instructions which, when read by the respective processor, cause the processor to receive the qMRI map or MR data describing an MR signal, and synthesize MR images as further detailed hereinabove. The program instructions can also cause the processor to train an artificial neural network as further detailed hereinabove. At least one of storage media 144 and 164 can store a database, such as, but not limited to, database 20.

In some embodiments of the present invention, a qMRI map or MR data describing an MR signal is transmitted to processor 132 by means of I/O circuit 134. I/O circuit 134 can receive the qMRI map or MR data via GUI 142, read it from storage medium 144, or receive it via network 140 from a remote, such as, but not limited to, computer 150.

Processor 132 receives the map or MR data, and synthesize one or more MR images as further detailed hereinabove. Computer 130 can display the synthesized MR images on GUI 142, or store them in storage medium 144. Alternatively, processor 132 can transmit the qMRI map or MR data over network 140 to server computer 150. Still alternatively, computer 150 can read the qMRI map or MR data from storage medium 164 or receive it via network from another source, such as, but not limited to, a cloud storage facility. Computer 150 can use the qMRI map or MR data to synthesize one or more MR images as further detailed hereinabove, and transmit the synthesized MR images to computer 130 over network 140. Computer 130 can receive the synthesized MR images from computer 150 and display them on GUI 142 or stores them in storage medium 144.

In some embodiments of the present invention a computing platform such as the computing platform shown in FIG. 4 is used for training a user to inspect and analyze MR images. In these embodiments, processor 132 displays on GUI 142 a training activation control, which cause the processor 132 to synthesize the MR image and display it on GUI 142 for training the user. A representative example of GUI 142 suitable for training the user to inspect and analyze MR images, is illustrated in FIG. 5.

GUI 142 can comprise an input control 502 allowing the user to provide the computer with a qMRI map or MR data. Typically, input control 502 includes is a dropdown menu that allows the user to select the qMRI map or MR data from a list prepared in advance, or a browse control allowing to user to select the a data file storing the qMRI map or MR data. Alternatively, the qMRI map or MR data can be received automatically from a storage medium or over a network as further detailed hereinabove in which case it is not necessary for input control 502 to allow selection of qMRI map or MR data. Also contemplated, are embodiments in which input control 502 allows the user to choose, e.g., by means of a radio button or the like, between an option of a user-selected qMRI map or MR data and the option of an automatic selection thereof.

Input control 502 can also allow the user to select tissue pathology to be mimicked on the synthesized MR image, and optionally and preferably also to select tissue type and/or the severity of the pathology. Input control 502 can also allow the user to select MRI settings, such as, but not limited to, at least one of a strength of the static magnetic field, gradient fields, MRI vendor, and pulse sequence. Indication regarding the various types of input provided by the user can be displayed in an information display area 520.

GUI 142 can also comprise a synthesis activation control 504, which may be in the form of a button control. Responsively to an activation of control 504 by the user, the computer synthesizes an MR image 508, as further detailed hereinabove, and displays it on a graphical area 506 of GUI 142. In some embodiments of the present invention the computer also displays an MR image 510 that corresponds to the unmodulated qMRI, for example, side-by-side to, or superimposed with, image 508. Optionally, the computer also displays a mark 512 on the synthesized image 508 at a location corresponding to the pathology. Mark can be displayed automatically or in response to activation of another activation control 514.

In some embodiments of the present invention the computer is configured to synthesize an ordered set of MR images and to display them one by one (e.g., each time in response to an activation of control 504 by the user). The ordered set of synthesized MR images can include synthesized MR images at which the visibility of synthesized pathology gradually increases or decreases, thereby allowing the user to be trained to identify also pathologies which are less pronounced over the background of normal tissue and are therefore harder to be identified. For example, the ordered set of synthesized MR images can include synthesized MR images at which the size of the pathology gradually increases or decreases, where the smallest size of the pathologies in the set is less than what would be distinguishable by the human observer, as further detailed hereinabove. Alternatively, or additionally, the ordered set of synthesized MR images can include synthesized MR images at which the severity of the pathology gradually increases or decreases, where the lowest severity of the pathologies in the set is less than the minimal level that can be distinguished by a human observer, as further detailed hereinabove.

The GUI 142 can also include one or more additional controls 516 (shown as a single control, for clarity of presentation) that may be in the form of button controls. For example, one of controls 516 can instruct the computer to clear the input at control 520 and/or the information at area 520, one of controls 516 can be an authentication control for authenticating the user, and the like.

While GUI 142 is schematically illustrated in FIG. 5 as an interface that includes a single screen, it is to be appreciated that GUI 142 can include more than one screen, such that at least two of the controls of areas of GUI 142 can be formed on different screens.

According to an aspect of some embodiments of the present invention there is provided a method of synthesizing a magnetic resonance (MR) image mimicking a tissue pathology. The method comprises: obtaining a quantitative MRI (qMRI) map of values of an MRI parameter;

accessing a computer readable medium storing a database having a plurality of entries each associating a database pathology to a database morphology; searching the database for an entry having a database pathology matching the tissue pathology; modulating values of the MRI parameter within a region of the qMRI map along a pattern selected based on a database morphology of the found database entry, thereby providing a modulated qMRI map; and generating an MR image based on the modulated qMRI map, thereby synthesizing the MR image with a synthesized pathology.

According to some embodiments of the invention, the method comprises generating the qMRI map.

According to some embodiments of the invention the qMRI map is generated based on an MR signal acquired from a subject.

According to some embodiments of the invention the subject is a healthy subject.

According to some embodiments of the invention each entry of the database also comprises database value or range of values of at least one MRI parameter that is associated with a respective database pathology, and the method comprises searching the database for an entry having a database pathology matching the tissue pathology, wherein the modulation of the values of the parameter is based on a database value or range of values of the found entry.

According to some embodiments of the invention the method comprises randomly selecting the region. According to some embodiments of the invention the region is predetermined.

According to some embodiments of the invention the region is less than 1 mm or less than 0.5 mm or less than 0.1 mm in length along its largest diameter.

According to some embodiments of the invention the modulation of the values of the MRI parameter is such as to provide, for each value, a modifying value that is within no more than ±16% or ±14% or ±12% or ±10% or ±8% or ±6% or less of an unmodified value of the value.

According to some embodiments of the invention the method comprises applying at least one morphology varying operation to the database morphology of the found entry.

According to some embodiments of the invention at least one of a type and an extent of the morphology varying operation is selected randomly.

According to some embodiments of the invention the database morphology corresponds to an orientation of muscle fibers.

According to some embodiments of the invention the database morphology corresponds to an orientation of neural fibers.

According to some embodiments of the invention the method comprises receiving input pertaining to a severity level of the tissue pathology, wherein the modulation of the values is based on the received severity level.

According to some embodiments of the invention the method comprises generating an simultaneous graphical output of the synthesized the MR image, and an MR image corresponding to the qMRI map prior to the modulation.

According to an aspect of some embodiments of the present invention there is provided a computer software product. The computer software product comprises a computer-readable medium in which program instructions are stored, which instructions, when read by a data processor, cause the data processor to receive a qMRI map of values of at least one MRI parameter and execute the method as delineated above and optionally and preferably as further detailed below.

According to an aspect of some embodiments of the present invention there is provided a method of training an artificial neural network. The method comprises: executing a method of synthesizing a magnetic resonance (MR) image mimicking a tissue pathology a plurality of times to respectively synthesize a plurality of MR images, each associated with at least one tissue pathology; feeding the artificial neural network with the synthesized MR images and the respective tissue pathologies, to obtain weight parameters for the artificial neural network; and storing the weight parameters in a computer readable medium; wherein the method of synthesizing the MR image mimicking the tissue pathology comprises the method as delineated above and optionally and preferably as further detailed below.

According to some embodiments of the invention the method comprises re-executing the synthesis of the MR image mimicking the tissue pathology an additional plurality of times to respectively synthesize an additional plurality of MR images, each associated with at least one tissue pathology; validating the weight parameters by feeding the artificial neural network with each of the additional plurality of synthesized MR images, and comparing an output of the artificial neural network with a respective tissue pathology; and generating a report indicative of the validation.

According to an aspect of some embodiments of the present invention there is provided a computer software product. The computer software product comprises a computer-readable medium in which program instructions are stored, which instructions, when read by a data processor, cause the data processor to receive a qMRI map of values of at least one MRI parameter and execute the method as delineated above and optionally and preferably as further detailed below.

According to an aspect of some embodiments of the present invention there is provided a computer software product for training a user. The computer software product comprises a computer-readable medium in which program instructions are stored, which instructions, when read by a data processor, cause the data processor to: display on a display device a graphical user interface (GUI) having a training activation control; automatically execute the method according to synthesize an MR image mimicking a tissue pathology by executing the method as delineated above and optionally and preferably as further detailed below, responsively to an activation of the control by the user; and generate a graphical output of the synthesized the MR image on the GUI.

According to some embodiments of the invention the program instructions, when read by a data processor, cause the data processor to synthesize an ordered set of MR images mimicking the tissue pathology, and to generate a graphical output separately for each of the MR images on the GUI.

According to some embodiments of the invention the set of MR images comprises synthesized MR images at which a visibility of the synthesized pathology gradually increases or decreases.

According to some embodiments of the invention the set of MR images comprises synthesized MR images at which a severity level of the synthesized pathology gradually increases or decreases.

According to some embodiments of the invention the set of MR images comprises synthesized MR images at which a size of the region gradually increases or decreases.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

The interpretation of medical images allows diagnosing pathologies. Traditionally, such interpretation is a time-consuming process performed manually by experts. A possible alternative to the manual approach is the use of artificial neural networks (e.g., DNN, CNN).

Construction and training of an artificial neural network requires use of large amounts of annotated data, which in turn, requires extensive number of costly scans and time-consuming expert annotation processes. Known in the art are data augmentation techniques, which increase the number of existing images using image transformations.

This Example describes a qMRI based technique that realistically simulates the manifestation of disease on MR images and at different levels of severity. This technique is based on incorporating the actual changes of the tissue biological properties into a biophysical signal model, thereby producing more faithful representation of diseases pathologies. The technique can also simulate a range of scanning protocols, scan settings, and vendors. Better training of artificial neural networks using this technique is useful for medical, research and/or educational centers.

In this Example, the generation of synthesized MR images is executed as follows. MR images are acquired from a cohort of healthy subjects. qMRI maps are computed from the MR images for physical MRI parameters (e.g., T₂, T₁, T₂*, diffusion values). Thereafter, the qMRI maps are modulated to introduce pathological changes. Preferably, the modulation comprises: (i) segmentation of a disease-prone regions-of-interest (ROIs), and (ii) mimicking pathological tissue changes by alteration of values in the qMRI maps obtained from the healthy subjects, within the ROIs.

The pathological changes can be incorporated into the qMRI maps in predefined spatial locations or randomly. The manner by which the qMRI map is modulated to introduce the pathology can be selected based on the neurobiological processes that underlie the appearance of these pathologies. For example, the location, shape, and size of synthetic brain lesions can follow neural tractography, measured using diffusion MRI. Alternatively, the addition of pathology can follow common radiologic manifestation of the disease. This can be done by segmentation of previously identified tissue pathologies, co-registration of the segmented pathologies and the qMRI maps, and optionally and preferably also imparting, e.g., randomly, changes to the pathologies' morphology, such as erosion, dilation, minoring, or changes to severity level and internal structure. The co-registration can be to any defined space. For example, for brain tissue, the co-registration can be to a standard brain coordinate system, such like the MNI system described at www(dot)nist(dot)mni(dot)mcgill(dot)ca/?page id=714.

The result of the modulation is one or more abnormal tissue (internal or external) morphologies, at specific locations and sizes. Synthetic MR images can then be generated based on the modulated qMRI maps using a qMRI signal model.

### Experimental

In this example T₂-weighted images were synthesized to mimic multiple sclerosis (MS).

MS is a chronic neurological disease affecting more than 2 million people worldwide. MS is characterized by demyelination of white matter WM in the central nervous system, visible on MRI. MS pathology is typically assessed on T₁ or T₂ weighted images (e.g., using MPRAGE or FLAIR) according to brain lesion load. Recent studies report the use of qMRI as an approach for diagnosing MS, allowing to identify pathology in normal-appearing white matter (NAWM). In this Example, the traditional diagnostic method was numerically evaluated using a two-alternative forced choice test (2AFC) and compared with an alternative qMRI-based tool.

### Methods

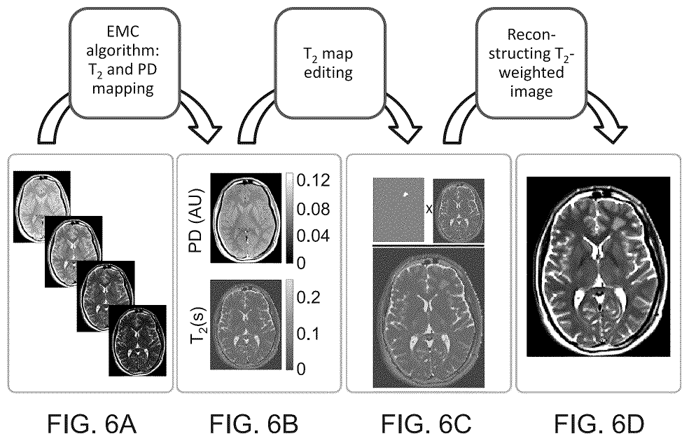

Axial brain images were acquired using a multi echo spin-echo (MESE) sequence. Imaging was done on a 3T Siemens Prisma scanner. Scan parameters included: N_{echoes}=20; TE/TR=10/2400 ms; in-plane resolution=1.5×1.5 mm², slice=3 mm, bandwidth=200 Hz/Px, N_{slices}=9 (raw data are shown in FIG. 6A). T₂ and proton density (PD) maps were generated using a pixel-wise fitting of the MESE data using the Echo-Modulation-Curve algorithm [Ben-Eliezer et al., Magn. Reson. Med. 73, 809-817 (2015)] (parametric maps are shown in FIG. 6B). The resulting T₂ maps were altered to simulate a MS related WM focal change in T₂ values (simulated MS lesions are shown in FIG. 6C). T₂ weighted images were reconstructed using a theoretical exponential decay model with the original PD map and the altered T₂ map (artificially lesioned T₂ images are shown in FIG. 6D).

T₂ weight images were simulated in 3 different echo times (TEs): 90, 70, and 40 ms, and 8 different lesion severity levels: 6, 9, 12, 15, 18, 21, 25 and 30% of change in T₂ value.

A trial was designed, according to the 2AFC method [Hanley et al., Radiology 143, 29-36 (1982)], to measure the human ability to detect pathological alteration of T₂ relaxation times via visual inspection of T₂ weighted images. During approximately 30 minutes, subjects were presented with a series of T₂ weighted brain images, each containing a single WM lesion with a predefined severity, mimicked by applying a predefined change in T₂ value of the qMRI map.

The trial consisted of two phases: a training phase and a test phase. In the training phase, the subject was presented with a pair of images of the same anatomy: one for reference, and another synthesized to mimic an artificial lesion. By comparing the two images, the subject learned the trial's typical lesion size, shape and location. During the test phase, subjects were shown a series of images from 3 different anatomical slices, and were requested to detect the existence location of lesions. The trial was repeated three times for three levels of T₂ weighting (three different echo times). Each trial consisted of 32 images with lesions at different levels of severity ranging from 0% to about 50%, where in this Example the severity is defined as the percentage of T₂ change in the lesions' ROIs, and 16 control images with no lesion. Subjects for the trial were a group of 33 neuroscience and medical students.

Trial data was analyzed to calculate the rates of true positives (correct detections of lesions), true negatives (correct identifications of images with no lesions), false positives (incorrect detections of nonexistent lesions) false negatives (missed lesions). Rates were then used to evaluate the subjects' performance (area under the receiver operating characteristic, AUC) in diagnosing MS pathology.

### Results

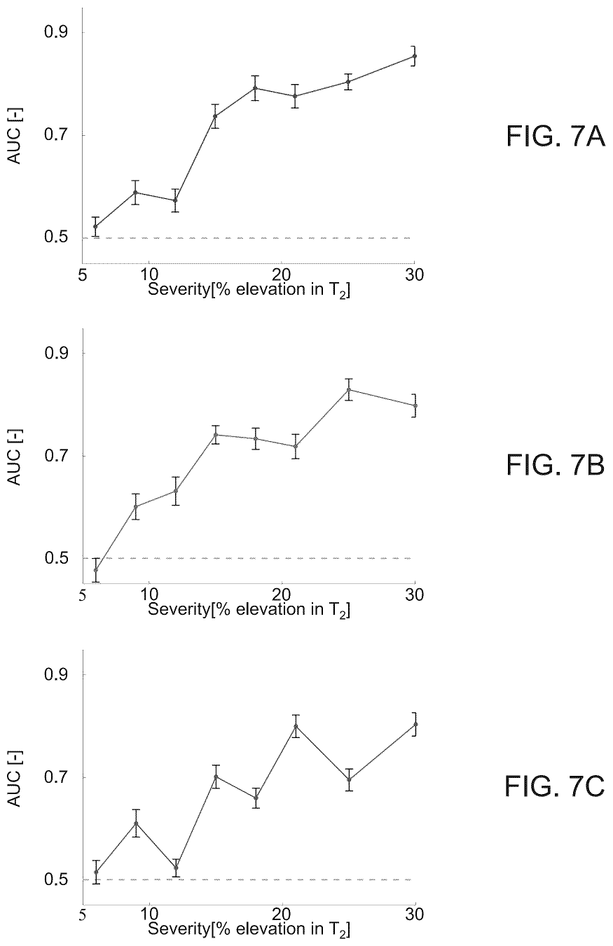

FIGS. 7A-C present the AUC detection rate as a function of lesion severity, for a group of 33 subjects. Shown are AUCs (mean±SE) for lesion detection as a function of the relative T₂ change at different TEs. The dashed line shows the AUC value equivalent to that of a random guess. FIG. 7A corresponds to TE of 90 ms, FIG. 7B corresponds to TE of 70 ms, and FIG. 7C corresponds to TE of 40 ms.

The results suggest that the tested population was able to detect pathological T₂ changes in brain WM tissue only for relative changes of 9% and above. AUCs for T₂ change of 6% were not significantly different than 0.5, meaning that the subjects did not perform better than a random guess for this value.

### Discussion

Quantitative assessment of pathology in NAWM was previously shown to be sensitive to about 5% change in the tissue T₂ value [Shepherd et al., Neurolmage Clin. 14, 363-370 (2017)]. This is based on comparing T₂ values in NAWM tissue of MS patients to heathy controls, and across different brain segment (e.g., caudate nucleus, body of corpus callosum). The visual assessment performed in this Example suggests that only changes of 9% and above are visually detectable.

The results presented in this Example thus suggest that the quantitative techniques of the present embodiments is a more powerful diagnostic tool.

### Example 2

Psychophysical Evaluation of Visual Vs. Computer-Assisted Detection of Brain Lesions: Pushing the Limits of Early Diagnosis

MRI based diagnosis is done by analyzing contrast-weighted images, e.g., T₁-, T₂-, or diffusion—weighted images. Pathologies appear as tissue abnormalities and are detected once they reach above a certain visual threshold. Computer assisted diagnosis (CAD) has been proposed as a way for achieving higher sensitivity to early pathology.

This Example compares conventional radiologic assessment of tissue abnormalities, to CAD based on a deep neural network. The studied model was multiple sclerosis (MS) lesions in the brain's white matter (WM). To that end, a psychophysical experiment was designed, in which radiologists and a neural network were asked to detect MS lesions on series of MR images. Lesions were artificially generated at predefined levels of severities, from subtle pathology to well-developed lesions by manipulating the tissue's transverse relaxation (T₂) time known to be the main source of contrast of MS pathology.

The results indicate that the odds ratios of identifying pathology in diseased tissue are smaller for visual radiologic inspection than for CAD, particularly when facing subtle pathologies (6-15% change in T₂ values), while a comparable performance is achieved at higher lesion severities (18-30% change in T₂ values). Most significant difference in performance was observed at severity of 6%, where radiologists' predictions were at a chance level (equal number of true-positive and false-positive predictions), while the corresponding CAD predictions produced statistically different diagnostic odds ratio (p<0.001).

This suggests that use of CAD can improve radiologic diagnosis by increasing the sensitivity to early pathology and the precision of disease follow-up.

### INTRODUCTION

Magnetic resonance imaging (MRI) is the most efficient modality for noninvasive imaging of soft-tissue pathologies. Traditionally, MRI diagnosis is done via time-consuming visual interpretation of contrast-weighted images, whose typical voxel size is in the range of ˜1-10 mm³. Most pathologies, however, emerge at the microscopic level and manifest radiologically only after reaching a certain level of severity, e.g., multiple sclerosis (MS), Parkinson's disease, Alzheimer's disease, or liver metastases. MRI based diagnosis is thus limited to abnormalities that spread over large enough volumes and above certain levels of severity. As a result, a significant effort has been invested throughout the last few decades in developing more sensitive tools, striving for earlier detection of tissue abnormalities and more precise assessment of treatment response.

Typical MR images are weighted by one or several physical properties of the tissue, e.g., relaxation times or diffusion, while also affected by external factors such as the receive or transmit coils sensitivity profiles, and inhomogeneity of the main magnetic field. A new and promising paradigm, termed quantitative MRI (qMRI), has emerged in the last decade, where instead of weighting images by a certain contrast, it is the actual value of the underlying physical property which is measured. This produces parametric maps, in which voxels hold meaningful numeric values pertaining to the tissue's microstructural architecture and chemical or biological composition, which, in turn, correspond to pathological processes. One of the main advantages of using qMRI is its improved sensitivity to tissue changes as exemplified by its ability to detect subtle pathology in normal appearing (NA) tissues. A second property is qMRI's ability to produce values that are invariant across scanners and scan setting, thereby facilitating longitudinal studies, and data sharing between medical centers.

Recently, several initiatives have been established, aiming to facilitate and advance the use of qMRI in the clinic (e.g., by the Radiological Society of North America and by the European Society of Radiology). Some of these include the use of computer assisted diagnosis (CAD) of parametric qMRI maps as a supplementary approach to visual interpretation of MR images. These include machine learning tools (Liu et al., 2019, Amer et al., 2019), voxel-based analysis (Piredda et al., 2020), region-of-interest analysis (Shepherd et al., 2017). The present embodiments also contemplate medical decision support systems. CAD can also be applied to contrast-weighted image data, albeit with lower robustness to data normalization, scaling, type of scanner, and scan parameters.

One of the elements for assessing the utility of qMRI based CAD tools is to test whether they can enhance the sensitivity of radiologic readings. This sensitivity can be tested with respect to various pathological features such as size, location, or severity. Several studies exist, evaluating the sensitivity of radiologic reading (Woo et al., 2006, Pikus et al., 2006, Drew et al., 2013, Altay et al., 2013, Brennan et al., 2018), or comparing human visual analysis vis-à-vis CAD algorithms where ground truth is obtained using other methods (e.g., retrospective diagnosis) (see, e.g., Liu et al. 2019).

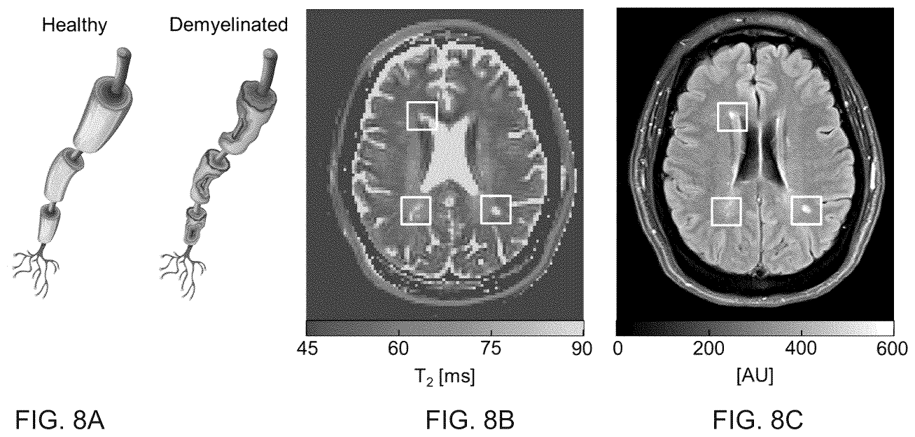

This Example describes a psychophysical experiment for comparing conventional radiologic assessment of tissue pathology to CAD using a deep learning neural network. The chosen disease model was multiple sclerosis (MS), which is characterized by inflammatory and demyelinating white matter (WM) lesions that manifest as hyperintensities on T₂-weighted MR images (see FIGS. 8A-C). Diagnosis of MS is based on the McDonald criteria, which, amongst other parameters, relies also on visual estimation of lesion load (Thompson et al., 2018, Filippi et al., 2016).

The psychophysical experiment was designed to include a series of T₂-weighted fluid attenuated inversion recovery (FLAIR) images, which were embedded with synthetic MS lesions at different levels of severity. These lesions were synthesized based on a biophysical model which links MS demyelinating processes to the ensuing changes in the tissue's T₂ relaxation times. In order to cover a wide range of pathological states, and to identify the minimal level of detectability, lesions were generated at eight levels of severity, from very subtle to highly visible lesions, reflecting a 6 to 30% change in the tissue's T₂ values. The level of detectability was then estimated for conventional visual assessment performed by a group of neuroradiologists, and for computer assisted diagnosis, performed using a supervised deep-learning neural network (DNN).

### Materials and Methods

Data collection

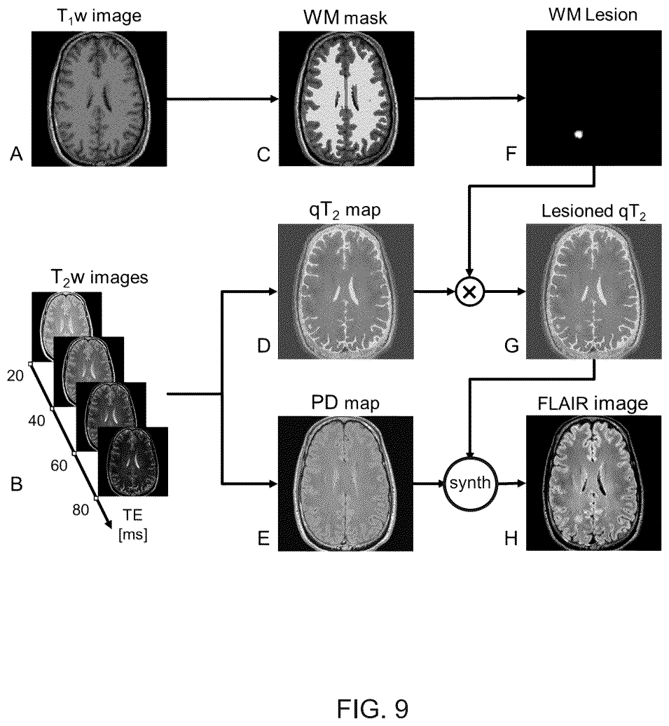

Data from 41 human volunteers was collected after obtaining informed consent and under the approval of the local ethics committee. Scans were performed on whole-body 3T MRI scanners (Prisma and Skyra, Siemens Healthineers Inc., Erlangen, Germany). Scans used a magnetization prepared rapid gradient echo (MPRAGE, FIG. 9, operation A) (Feinberg et al., 1985), multi-echo spin-echo (MESE, FIG. 9, operation B) (Hajnal et al., 1992) and FLAIR (Lesjak et al., 2018). Scan parameters are given in Table 2.1, below. Additional data was imported from a public MS patients MRI dataset (Ben-Eliezer et al., 2015).

Table 2.1 provides datasets, protocols, and experimental parameters, used for data acquisition. Data collected can be divided to 3 datasets. Dataset 1 consisted of three healthy subjects (one female). Dataset 2 consisted of 8 healthy subjects (three females). Scans for datasets 1 and 2 were performed on a whole-body 3T MRI scanner (Siemens Prisma). Dataset 3 consisted of 33 healthy subjects, collected on a whole-body 3T MRI scanner (Siemens Skyra).

TABLE 2.1

| | Protocol | | | | | | |
|---|---|---|---|---|---|---|---|
| | MESE | | | MPRAGE | | | FLAIR |
| Parameter | Dataset 1 | Dataset 2 | Dataset 3 | Dataset 1 | Dataset 2 | Dataset 3 | Datasets 1 & 2 |
| Dimensionality | 2D | 2D | 2D | 3D | 3D | 3D | 2D |
| TR [sec] | 3 | 2 . . . 5.15 | 2.5 | 2.4 | 1.75 | 2.1 | 8 |
| TI [ms] | — | — | — | 1000 | 900 | 900 | 2370 |
| TE [ms] | 10 | 10 . . . 15 | 12 | 2.8 | 2.6 | 2.3 | 10.1 |
| Echo spacing [ms] | 10 | 10 . . . 15 | 12 | — | — | — | 10.1 |
| Echo train length | 15 . . . 20 | 7 . . . 20 | 10 | 1 | 1 | 1 | 16 |
| In-plane resolution [mm²] | 0.8² . . . 0.9² | 0.6² . . . 1.7² | 1.7² | 0.9² | 1² | 1² | 0.7² |
| Slice thickness [mm] | 3 | 1 . . . 3 | 3 | 0.9 | 1 | 1 | 4 |

### Post Processing: Generation of Parametric qT₂ Maps and WM Masks

MESE data was used to generate quantitative T₂ (qT₂, FIG. 9, operation D) and proton density (PD, FIG. 9, operation E) maps using the echo modulation curve (EMC) algorithm (Fischl et al., 2002). Fitting procedures were programmed in-house using C++ and MATLAB (The MathWorks Inc., Natick, MA). Volumetric segmentation of the entire WM was done on MPRAGE scans using Freesurfer software (Greve et al., 2009). Registration of the resulting WM mask to qT₂ and PD maps were performed using Freesurfer (DAW, 1962) (FIG. 9, operation C).

### Generation of Synthetic Lesions

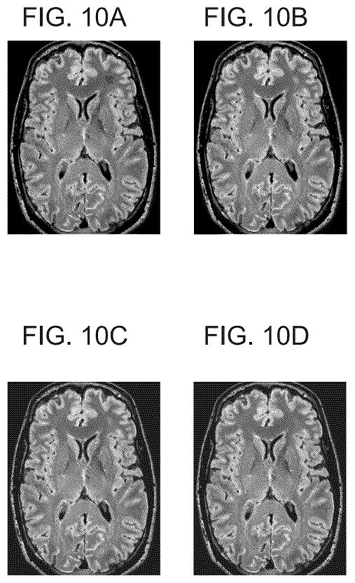

Synthetic lesions were embedded into 35 qT₂ maps of healthy brains. Lesions' location and shape were determined using classic image processing tools. First, a center focal point was randomly selected within the WM mask. Voxels within a radius of 1 cm around the focal point were then chosen randomly, and the lesion's area was calculated as the convex hull of the chosen points (FIG. 9, operation F). MS pathology was simulated by elevating the qT₂ values within the lesion's area to one of eight predetermined severity levels in the range of 6, 9, 12, 15, 18, 25 and 30%. Elevation of values was applied in a pseudo spatially centric manner where a maximal increase was applied at the lesion center, which decreased to zero change towards the edges. More examples of simulated lesions are shown in FIGS. 10A-D.

### Conversion to Synthetic FLAIR Images

Modified qT₂ maps (FIG. 9, operation G) were used to generate the synthetic FLAIR images that were used in the psychophysical experiment (FIG. 9, operation H). Conversion was performed using an analytic signal model for the acquisition of FLAIR signal on an MRI scanner, abbreviated in FIG. 9 as “synth.” The Parameters for this model were optimized to resemble the corresponding acquired FLAIR scans. Ensuing FLAIR images were finally examined by an expert neuroradiologist with 10 years of experience, validating the synthetic FLAIR contrast and the appearance (location and size) of the simulated MS lesions. Further details regarding this model are provided in Example 3, below.

### Two Alternative Forced Choice Psychophysical Experiment for Lesion Detection

Two alternative forced choice (2AFC) psychophysical experiment was designed to assess the efficiency of conventional radiologic detection of tissue pathology. 25 radiologists (10 females), 29 to 65 years old (39.4±9.2), with 1 to 35 years of experience (8.9±9.8) were recruited from two large hospitals in Israel, to participate in the experiment. All participants had normal or corrected-to-normal vision and received 50 NIS (˜16 USD) for participation. The experiment was approved by Tel Aviv University ethics committee. Informed consent was obtained from all participants. Stimuli for the experiment consisted of 48 two-dimensional FLAIR images, having a constant spatial resolution (256×256), and taken from the same supratentorial brain region. Image series contained 32 images with a single, oval, hyperintense lesion, and 16 images that were unedited and lesion free.

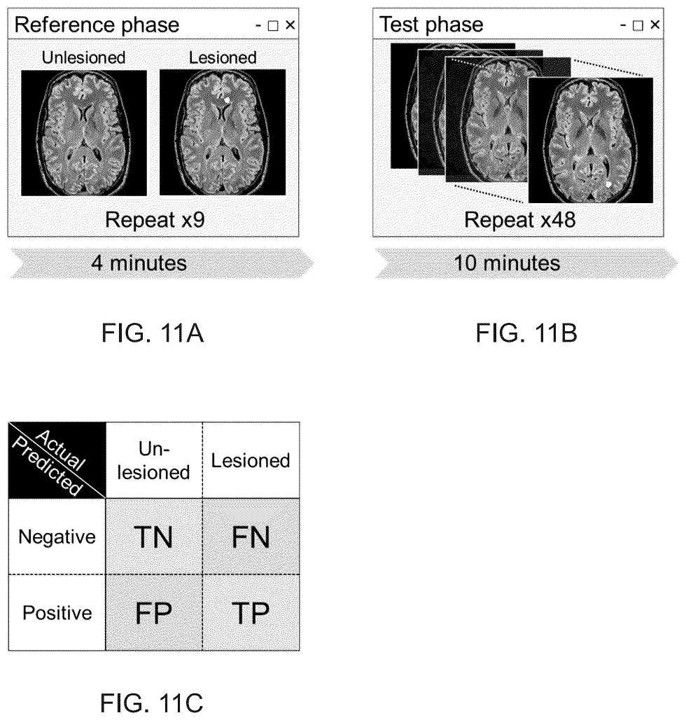

A diagram of the psychophysical experiment protocol is illustrated in FIGS. 11A-C. Prior to the experiment, participants were informed on the distribution of lesioned scans. The experiment itself begun with a practice phase consisting of 9 images, out of which 6 were lesioned, and providing feedback on the detection accuracy. During the experiment, participants were shown the series of 48 FLAIR images and asked to point out lesions. Images were present on the screen for 10 s each, while blank images were shown for 400 ms between each FLAIR images to reduce afterimage effect. Experiment was split into two phases, each containing 24 images, and separated by an elective break of 1-5 min. Participants were allowed to skip images (i.e., shorten the 10 s period image), and their selections and response time were recorded.

### Computer Assisted Detection of Lesions

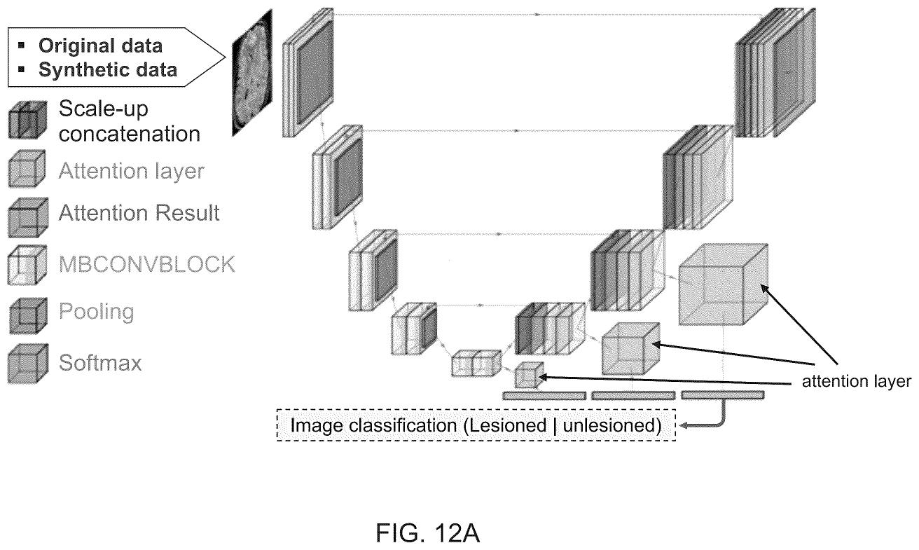

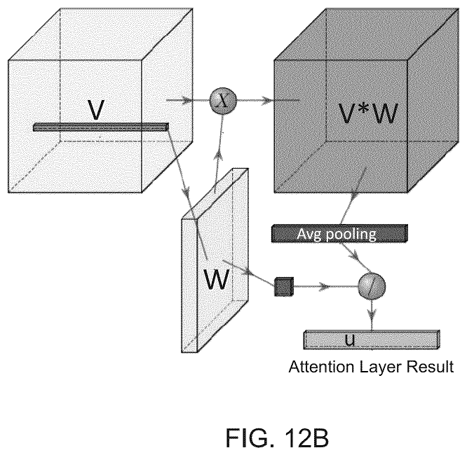

To assess CAD based detection of lesions, the same series of images comprising the psychophysical experiment were binarily classified by a convolutional neural network (CNN). The network architecture was inspired from Y-Net (Tan et al., 2019) with an EfficientNet (Cox, 1958) backbone, which included attention layers, allowing the extraction of lesions' locations, and reducing overfitting. The network architecture is illustrated in FIGS. 12A-B and is described in greater detail in Example 3. The attention weight mask was regularized by an innovative scheme (see Example 3, EQ. 3.2) which affected the amount of focus on the lesions area in comparison to other areas in the image. Pre training of the network was done using MS patients' FLAIR images from a published dataset (Ben-Eliezer, 2015). Training and validation were performed using the synthetically generated FLAIR images (N_{Total}=9600), containing images of healthy anatomy (N_{Healthy}=3200) and of simulated lesions (N_{Lesions}=6400). The entirety of data used for training was not included in the psychophysical test. Implementation of the network was done in Python using the PyTorch library, and the training process was performed on a standard desktop PC, equipped with an Nvidia GeForce GTX 1080 T₁ GPU.

### Evaluation of Computer Assisted Vis-à-Vis Radiologic Detection of Brain Lesions

To evaluate the performance of CAD-based detection versus conventional radiological detection of brain lesions, a series of statistical tests were performed using SPSS Statistics software (version 24, IBM) and using Matlab (Mathworks Inc., Natic, MA, USA). Logistic regression was used to determine the change in accuracy as a function of experts' years of experience and lesion severity (% of change in T₂ values). Cohen's kappa coefficient was used to evaluate the agreement between the two detection methods:

$$κ = \frac{{P}_{o} - {P}_{e}}{1 - {P}_{e}} . \qquad ( EQ . 2.1 )$$

Diagnostic odd ratios (OR) were calculated for CAD and for radiologic detection, according to the formula:

$$OR = \frac{TP \cdot TN}{FP \cdot FN} , \qquad ( EQ . 2.2 )$$

and the two approaches were compared using Z-test for log(OR):

$$Z - score = \frac{log ( {OR}_{1} ) - log ( {OR}_{2} )}{\sqrt{{SD}_{ln ( {OR}_{1} )}^{2} + {SD}_{ln ( {OR}_{1} )}^{2}}} , \qquad ( EQ . 2.3 )$$

where SD_{log(OR)}² is the variance of the logarithm of the diagnostic odd ratio, defined as:

SD_{log(OR)}²=TP⁻¹+TN⁻¹+FP⁻¹+FN⁻¹ (EQ. 2.4)

### Results

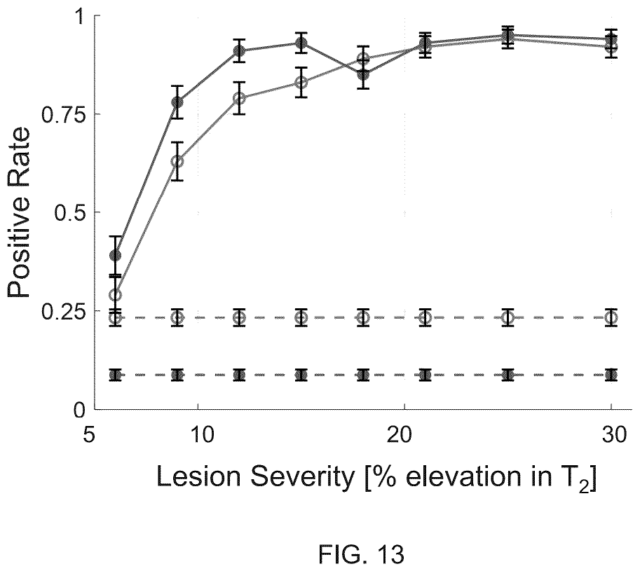

Radiologists took an average of 5.6±3.4 seconds to analyze each image, while the overall duration of the psychophysical experiment was 7:42±1:26 minutes. FIG. 13 presents the efficiency of radiologic and computer assisted detection of lesions, including true positive (TP) and false positive (FP) rates per severity level. TP rates (1−FN, solid lines) for both radiologists and CAD increase with lesion severity. TP rate for CAD was significantly higher than that of radiologists (p-value<0.05) at middle-low severity levels of 9-15% elevation in T₂, and comparable (p-value>0.05) at 6% and at levels of 18-30%. WM lesions that are obvious to radiologists manifest a significantly higher elevation of ≥35% change in T₂ values compared to homologues regions in healthy controls. The FP rate for CAD was significantly lower than that of radiologists (8.75% vs. 23.3% respectively). At very low severity level (6% elevation in T₂), the radiologist TP rate was not significantly different from their FP rates (p-value>0.05), indicating that the diagnosis was done at a chance level. More specifically, the lack of difference between TP and FP rates implies that the average evaluator has similar probability of classifying the image as lesioned or unlesioned, regardless of the underlying ground truth.

### Agreement Between Radiologists and CAD

Cohen's kappa scores for interobserver agreement between visual and computer assisted detection indicated moderate agreement between the two approaches across all lesion severities. While some level of agreement is expected when using two different modalities, this score suggests no internal bias of the data, which would have biased both methods similarly Specific kappa scores were calculated for several classifications of the data: considering only positive/negative binary decision produced a κ=0.41; considering a binary decision but separately for each severity level produced kappa scores of κ<0.48; considering a four-way diagnosis (TP, TN, FP, FN) across all severity levels, resulted in a score of κ=0.52; and lastly, considering a four-way diagnosis but separately for each severity level, produced κ<0.55. Detection performance for radiologists and CAD, along with the full list of kappa scores, are delineated in Tables 2.2 and 2.3.

Table 2.2 relates to the agreement between CAD and radiologists. Actual and predicted classification of lesions for all experiments. Ground truth classification can be either negative (N, two left columns) for images without lesions or positive (P, columns on the right) for images with lesions.

TABLE 2.2

| | Actual Classification | | | | | | | | | | | | | | | | | |
|---|---|---|---|---|---|---|---|---|---|---|---|---|---|---|---|---|---|---|
| | N | | P | | | | | | | | | | | | | | | |
| | Lesion Severity [% elevation in T₂] | | | | | | | | | | | | | | | | | |
| | 0 | | 6 | | 9 | | 12 | | 15 | | 18 | | 21 | | 25 | | 30 | |
| | P | N | P | N | P | N | P | N | P | N | P | N | P | N | P | N | P | N |
| Positive | 11 | 24 | 20 | 19 | 56 | 22 | 77 | 14 | 79 | 14 | 82 | 3 | 88 | 5 | 90 | 5 | 88 | 6 |
| Negative | 82 | 283 | 9 | 52 | 7 | 15 | 2 | 7 | 4 | 3 | 7 | 8 | 4 | 3 | 4 | 1 | 4 | 2 |

Table 2.3 proved kappa scores for radiologists and for CAD-based lesion detection. Rightmost column contains the overall kappa score calculated across all lesion severity levels. Top: prediction kappa was calculated while considering only positive/negative binary decision. Bottom: prediction kappa was calculated while considering a four-way diagnosis.

TABLE 2.3

| | Lesion Severity [% elevation in T₂] | | | | | | | | | |
|---|---|---|---|---|---|---|---|---|---|---|
| | 6 | 9 | 12 | 15 | 18 | 21 | 25 | 30 | 0 | Overall |
| Prediction kappa | 0.16 | 0.32 | 0.42 | 0.42 | 0.46 | 0.47 | 0.48 | 0.47 | 0.47 | 0.41 |
| 4 categories kappa | 0.48 | 0.48 | 0.52 | 0.51 | 0.54 | 0.54 | 0.54 | 0.54 | — | 0.52 |

### Odds Ratios Comparison Per Severity Level

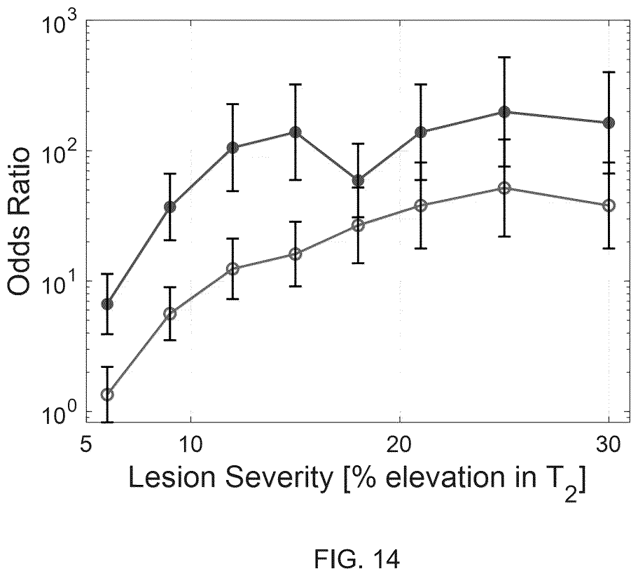

Overall diagnostic odds ratios (ORs) for radiologists and for CAD were 11.5 and 50.5, respectively. The CAD OR was statistically higher than radiologists' OR (p-value<0.001). FIG. 14 presents the OR values for radiologists and for CAD across the lesions' severity levels. All values exhibited a consistent correlation to lesion severity, except for a single CAD OR value at severity level of 18%, which we consider an outlier of the experiment. Analyzing each severity level separately, ORs for CAD were significantly higher than ORs for radiologists for the four lowest severity levels (6, 9, 12, 15% elevation in T₂), and comparable for higher levels. Notably, the radiologists OR for the first severity level (6% elevation in T₂) was not statistically significantly higher than 1. This is consistent with the similarity between the FP and TP rates for severity level of 6% (23.3% and 29% respectively in FIG. 13), indicating that this level of severity is below the threshold of visual detection in the experimental settings of this study.

### Trends in Radiologists' Accuracy Per Years of Experience and Per Severity Level

Logistic regression was performed for the radiologists' diagnostic accuracy per years of experience and per lesion severity. Diagnostic accuracy is defined as (TP+TN)/(TP+TN+FP+FN). Based on the regression model the radiologists' error rate decreased by 1.9% for each year of experience and decreased by 2.5% per one percent change in T₂. Both findings were significant with p-value<0.001.

### DISCUSSION

This Example compared the diagnostic performance of radiologists and of a neural-network based CAD tool. To that end, a diagnostic psychophysical experiment was designed for the radiologists, and later given as input to the CAD tool. Results show that the selected CAD tool outperforms radiologists at low severity levels, and provides comparable diagnostic capability at high severity levels. This suggests that the CAD tool of the present embodiments can serve as a guide to radiologic analysis, particularly for early diagnosis of subtle tissue abnormalities and when processing large amounts of data.

The psychophysical experiment performed in this Example was designed to match clinical settings as closely as possible. This included simulating realistically looking lesions on standard FLAIR MRI images, followed by authenticating their morphology and location by a trained neuroradiologist with 10 years of experience.

This Example demonstrates the advantage of using CAD based detection of MS lesions. This Example demonstrates that experts' time can be saved by embedding new, automated, tools for detecting abnormalities in medical images. The CAD based detection of the present embodiments can lead to a more scalable, accessible, and precise diagnosis of diseases, while improving the throughput of radiologic reading.

### Example 3

This Example provides supplementary Information to the experiments described in Example 2.

### Data Collection: Acquisition Parameters

Three datasets were collected. Dataset #1 consisted of three healthy subjects (one female), dataset #2 consisted of 8 healthy subjects (3 females); and dataset #3 consisted of 33 healthy subjects (15 females). Subjects for datasets #1 and #2 were scanned on a whole-body 3T MRI scanner (Siemens Prisma), while subjects for datasets #3 were scanned on a whole-body 3T MRI scanner (Siemens Skyra). Scan parameters are available in Table 2.1, above. Scans from dataset #1 were used for the psychophysical experiment. Scans from datasets #2 and #3 were used for training of the neural network CAD tool tested in this work.

### Generation of Synthetic FLAIR Images

T₂ weighted MESE images were converted to a FLAIR contrast, commonly used in radiologic diagnosis of MS pathology. FLAIR images mix T₁ contrast due to the inversion recovery module, and T₂ contrast due to the turbo spin-echo (TSE) acquisition scheme. Since only T₂ maps were available for the data, the missing T₁ values were estimated using a linear regression of the correlation between T₂ and T₁ values in the brain, based on quantitative relaxation atlases published by Piredda et. al. (G. F. Piredda, et al., Magnetic Resonance in Medicine 83, 337-351 (2020)).

FLAIR images were synthesized using a signal model (Herskovits et al., American Journal of Roentgenology 176, 1313-1318 (2001), Woo et al., Radiology 241, 206-212 (2006), Pikus et al., et al., Radiology 239, 238-245 (2006)), incorporating the above two relaxation mechanisms as well as the sampling scheme used in a FLAIR sequence (Hendrick et al., Magnetic Resonance Imaging 2, 193-204 (1984), Hennig et al., Magnetic Resonance in Medicine 3, 823-833 (1986), Hajnal et al., Journal of Computer Assisted Tomography 16, 841-844 (1992)).

$${S}_{FLAIR} = PD ( 1 - ( 1 - cos ( FA ) ) exp ( \frac{- TI}{{T}_{1}} ) ) exp ( \frac{- {TE}_{i}}{{T}_{2}} ) , i = 1 … TF \qquad ( EQ . 3.1 )$$

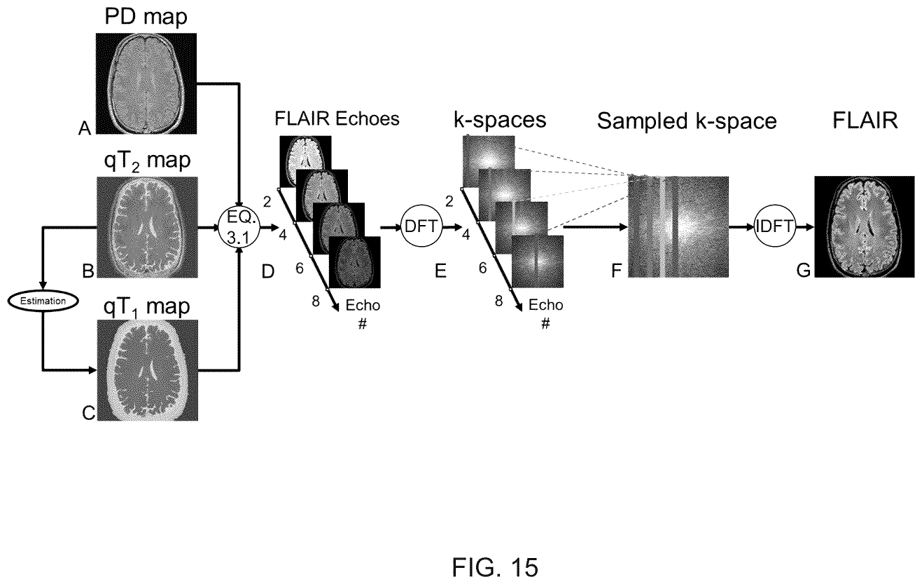

In EQ. 3.1, TI is the inversion time, FA is the excitation flip-angle, TE is the echo time, and TF is the turbo factor. The values of these parameters were all extracted from the scan settings. The synthesis of FLAIR images used the T₂ and PD maps as input and was done using the following steps (see FIG. 15). A T₁ map was estimated according to Piredda et. al. supra. The T₂ and PD maps were then used to generate a series of T₂-weighted images at increasing TE's I(x, y, TE), and an inversion-recovery T₁ weighting was applied on the series of T₂ weighted images according to EQ. 3.1I(x, y,TE,TI). The new series of images was transferred to k-space by a Fourier transform: I(x, y, TE, TI)→î(kₓ,k_{y},TE,TI). The FLAIR image k-space data was generated by extracting k-space lines from Î where k₁ was extracted from the image of the first TE Î(TE), k₂ was extracted from the second TE image Î(2×TE), and so forth until the turbo factor was reached for k_{TF}. The next series of TF k-space lines were then extracted again from the images of the first to last image until the entire k-space was full. The final FLAIR image was then generated using an inverse Fourier transform of the generated k-space data.

Simplifications present in this approach are the assumption of long TR (TR»T₁»T₂), short echo times (TE×ETL«T₁) and negligible effect of stimulated echoes. Those assumptions hold for the sequence simulated by the model due to the choice of parameters (TR=8000 ms, TE×ETL=162 ms). The effect of stimulated echoes can be taken into account using more complex models (e.g., extended phase graphs (J. Hennig, Journal of Magnetic Resonance 78, 397-407 (1988)), Bloch simulations (Ben-Eliezer, 2015)), which heretofore have not been used for synthetic MRI.

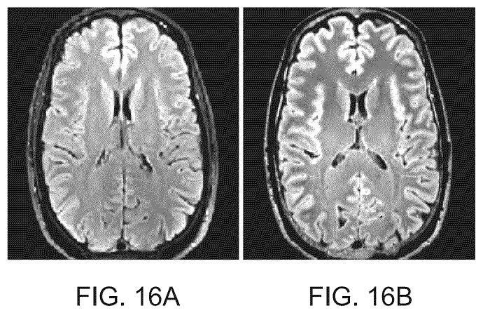

Synthetic FLAIR images of the brains were validated by an expert radiologist with 10 years of experience. Synthetic and conventional FLAIR images are given in FIGS. 16A and 16B, where FIG. 16A shows an axial slice of the brain acquired using a FLAIR protocol, and FIG. 16B shows a synthetic FLAIR image generated from a qT₂ map of the same slice using the signal model described in EQ. 2.1.

A small bright rim artifact is seen in the border of the gray matter and cerebrospinal fluid. This artifact is common in the literature of synthetic FLAIR contrasts and is due to partial volume effect (Hagiwara et al., Investigative Radiology 52, 647-657 (2017)). Although appearing in all FLAIR images, this artifact is located far from the locations of the synthetic MS lesions and was thus considered to not interfere with the psychophysical test.

### Generation of the Neural Network Used for CAD

The architecture utilized for this work is inspired from Y-Net (Mehta et al., in International Conference on Medical Image Computing and Computer-Assisted Intervention, (2018), pp. 893-901) model, with an EfficientNet backbone (Tan et al., in Proceedings of the 36th International Conference on Machine Learning, Proceedings of Machine Learning Research., K. Chaudhuri, R. Salakhutdinov, Eds. (PMLR, 2019), pp. 6105-6114).

The architecture of the neural network used for CAD is illustrated in FIG. 12A. Efficient-net blocks are used also in the decoder. Attention blocks were integrated in the last layer of the backbone and in the end of every block before the next scale up. Linear classification layer was added on output of attention layers. The input to the network was a series of original and synthetic brain MRI images. The output of the network was a binary classification of images as lesioned or unlesioned. The architecture of the attention layer (marked by arrows in FIG. 12A) is illustrated in FIG. 12B. The Input to attention layer network is network layer (V). Convolution 1×1 is performed on V to generate attention map (W), which provides higher values in a lesioned area. The convolution is followed with batch normalization and Sigmoid operation to extract probabilistic [0-1] values in W. The weight layer, W, is then multiplied with network layer V to generate a Weighted network layer (V×W). Average pooling, followed by division, provides the Attention result vector u (uᵢ=Σw×vᵢ/Σw, where i is the slice index). Thus, u denotes the spatial weighted mean of network layer V with weights W. The output attention result u, is used for the final classification decision.

The network was able to amplify the lesion area and attenuate lesion-free areas owing to the added loss. In addition, a classical binary cross entropy loss was used for classification.

The loss added on the mask layer of the attention block (FIG. 12A) forces most of the energy in the lesion by minimizing ρ ratio loss:

$$ρ = {( \sqrt{\frac{{{\sum }_{\{ n , m \}} {x}_{\{ n , m \}}^{2} - {\sum }_{\{ n , m ∈ Ω \}} {x}_{\{ n , m \}} )}^{2} + ϵ}{{\sum }_{\{ n , m ∈ Ω \}} {x}_{\{ n , m \}}^{2} + ϵ}} - δ )}^{+} \qquad ( EQ . 3.2 )$$

where Ω is the set of indices in the lesion's bounding box, x_{{n,m}} is a pixel in the mask layer with the indices (n, m), ϵ>0 prevents divergence and δ is a regularization parameter that determines how soft is the loss. The symbol (•)⁺ indicates a rectified linear unit (ReLU).

When δ→0, the loss force all the energy of the mask layer to locate in the lesion. When δ→1, the loss force allow some freedom to the network mask to search out of the lesion area. The ReLU in the loss prevents the loss p from being negative when δ>0.

The back bone used for this model was pretrained using non-medical images from ImageNet (Deng et al., in 2009 IEEE Conference on Computer Vision and Pattern Recognition, (IEEE, 2009), pp. 248-255). MR images used for the network were MS patients' FLAIR scans from a published dataset (Lesjak et al., Neuroinformatics 16, 51-63 (2018)), containing 30 different patients, for pretraining, and Synthetic FLAIR generated from datasets 2 and 3 (Table 2.1), for training and validation. Images shown to the network were normalized and concatenated with a matching grid on the x and y axes. The model was trained using the RMSProp optimizer. The network was written in Python and used the Pytorch library. The training process was performed on a standard PC with an Nvidia GeForce GTX 1080 Ti GPU.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

Reich, D. S., Lucchinetti, C. F. & Calabresi, P. A. Multiple Sclerosis. N. Engl. J. Med. 378, 169-180 (2018).
Filippi, M. et al. MRI criteria for the diagnosis of multiple sclerosis: MAGNIMS consensus guidelines. Lancet Neurol. 15, 292-303 (2016).
Shepherd, T. M. et al. New rapid, accurate T2 quantification detects pathology in normal-appearing brain regions of relapsing-remitting MS patients. Neurolmage Clin. 14, 363-370 (2017).
Gracien, R.-M. et al. Multimodal quantitative MRI assessment of cortical damage in relapsing-remitting multiple sclerosis. J. Magn. Reson. Imaging 44, 1600-1607 (2016).
Hagiwara, A. et al. Utility of a Multiparametric Quantitative MRI Model That Assesses Myelin and Edema for Evaluating Plaques, Periplaque White Matter, and Normal-Appearing White Matter in Patients with Multiple Sclerosis: A Feasibility Study. Am. J. Neuroradiol. 38, 237-242 (2017).
Woo, J. H., Henry, L. P., Krejza, J. & Melhem, E. R. Detection of Simulated Multiple Sclerosis Lesions on T2-weighted and FLAIR Images of the Brain: Observer Performance. Radiology 241, 206-212 (2006).
Liu, X. et al. A comparison of deep learning performance against health-care professionals in detecting diseases from medical imaging: a systematic review and meta-analysis. Lancet Digit. Heal. 1, e271-e297 (2019).
Cook, T. S. Human versus machine in medicine: can scientific literature answer the question? Lancet Digit. Heal. 1, e246—e247 (2019).
Hanley, J. A. & McNeil, B. J. The meaning and use of the area under a receiver operating characteristic (ROC) curve. Radiology 143, 29-36 (1982).
Ben-Eliezer, N., Sodickson, D. K. & Block, K. T. Rapid and accurate T 2 mapping from multi-spin-echo data using Bloch-simulation-based reconstruction. Magn. Reson. Med. 73, 809-817 (2015).
E. J. R. van Beek, et al., Value of MRI in medicine: More than just another test? Journal of Magnetic Resonance Imaging 49, e14—e25 (2019).
F. Lüsebrink, A. Sciarra, H. Mattern, R. Yakupov, O. Speck, T1-weighted in vivo human whole brain MRI dataset with an ultrahigh isotropic resolution of 250 μm. Scientific Data 4, 170032 (2017).
A. J. Thompson, et al., Diagnosis of multiple sclerosis: 2017 revisions of the McDonald criteria. The Lancet Neurology 17, 162-173 (2018).
W. Poewe, et al., Parkinson disease. Nature Reviews Disease Primers 3, 17013 (2017).
K. A. Johnson, N. C. Fox, R. A. Sperling, W. E. Klunk, Brain Imaging in Alzheimer Disease. Cold Spring Harbor Perspectives in Medicine 2, a006213-a006213 (2012).
A. D. Hardie, et al., Diagnosis of liver metastases: value of diffusion-weighted MRI compared with gadolinium-enhanced MRI. European Radiology 20, 1431-1441 (2010).
F. Bloch, Nuclear Induction. Physical Review 70, 460-474 (1946).
H. Y. Carr, E. M. Purcell, Effects of Diffusion on Free Precession in Nuclear Magnetic Resonance Experiments. Physical Review 94, 630-638 (1954).
E. O. Stejskal, J. E. Tanner, Spin Diffusion Measurements: Spin Echoes in the Presence of a Time-Dependent Field Gradient. The Journal of Chemical Physics 42, 288-292 (1965).
P. B. Roemer, W. A. Edelstein, C. E. Hayes, S. P. Souza, 0. M. Mueller, The NMR phased array. Magnetic Resonance in Medicine 16, 192-225 (1990).
H. Chang, J. M. Fitzpatrick, A technique for accurate magnetic resonance imaging in the presence of field inhomogeneities. IEEE Transactions on Medical Imaging 11, 319-329 (1992).
N. Ben-Eliezer, “Advances in Signal Processing for Relaxometry” in Quantitative Magnetic Resonance Imaging, (2020), pp. 123-147.
I. Seroussi, D. S. Grebenkov, O. Pasternak, N. Sochen, Microscopic interpretation and generalization of the Bloch-Toney equation for diffusion magnetic resonance. Journal of Magnetic Resonance 277, 95-103 (2017).
A. Ceccarelli, et al., Deep gray matter T2 hypointensity is present in patients with clinically isolated syndromes suggestive of multiple sclerosis. Multiple Sclerosis Journal 16, 39-44 (2010).
A. Hagiwara, et al., Linearity, Bias, Intrascanner Repeatability, and Interscanner Reproducibility of Quantitative Multidynamic Multiecho Sequence for Rapid Simultaneous Relaxometry at 3 T. Investigative Radiology 54, 39-47 (2019).
R.-M. Gracien, et al., How stable is quantitative MRI?—Assessment of intra- and inter-scanner-model reproducibility using identical acquisition sequences and data analysis programs. Neurolmage 207, 116364 (2020).
K. E. Keenan, et al., Recommendations towards standards for quantitative MRI (qMRI) and outstanding needs. Journal of Magnetic Resonance Imaging 49, e26-e39 (2019).
L. G. Kessler, et al., The emerging science of quantitative imaging biomarkers terminology and definitions for scientific studies and regulatory submissions. Statistical Methods in Medical Research 24, 9-26 (2015).
N. M. deSouza, et al., Validated imaging biomarkers as decision-making tools in clinical trials and routine practice: current status and recommendations from the EIBALL* subcommittee of the European Society of Radiology (ESR). Insights into Imaging 10, 87 (2019).
R. Amer, J. Nassar, D. Bendahan, H. Greenspan, N. Ben-Eliezer, “Automatic Segmentation of Muscle Tissue and Inter-muscular Fat in Thigh and Calf MRI Images” in Medical Image Computing and Computer Assisted Intervention—MICCAI 2019, (2019), pp. 219-227.
G. F. Piredda, et al., Quantitative brain relaxation atlases for personalized detection and characterization of brain pathology. Magnetic Resonance in Medicine 83, 337-351 (2020).
L. Pikus, et al., Artificial Multiple Sclerosis Lesions on Simulated FLAIR Brain MR Images: Echo Time and Observer Performance in Detection. Radiology 239, 238-245 (2006).
T. Drew, M. L.-H. VO, J. M. Wolfe, The Invisible Gorilla Strikes Again. Psychological Science 24, 1848-1853 (2013).
E. E. Altay, et al., Reliability of Classifying Multiple Sclerosis Disease Activity Using Magnetic Resonance Imaging in a Multiple Sclerosis Clinic. JAMA Neurology 70, 338 (2013).
P. C. Brennan, et al., Radiologists can detect the ‘gist’ of breast cancer before any overt signs of cancer appear. Scientific Reports 8, 8717 (2018).
T. S. Cook, Human versus machine in medicine: can scientific literature answer the question? The Lancet Digital Health 1, e246-e247 (2019).
O. Commowick, et al., Objective Evaluation of Multiple Sclerosis Lesion Segmentation using a Data Management and Processing Infrastructure. Scientific Reports 8, 13650 (2018).
T. Meng, et al., The diagnostic performance of quantitative mapping in breast cancer patients: a preliminary study using synthetic MRI. Cancer Imaging 20, 88 (2020).
M. Filippi, et al., MRI criteria for the diagnosis of multiple sclerosis: MAGNIMS consensus guidelines. The Lancet Neurology 15, 292-303 (2016).
B. Efron, “Bootstrap Methods: Another Look at the Jackknife” in Breakthroughs in Statistics, (1992), pp. 569-593.
S. Dargan, M. Kumar, M. R. Ayyagari, G. Kumar, A Survey of Deep Learning and Its Applications: A New Paradigm to Machine Learning. Archives of Computational Methods in Engineering 27, 1071-1092 (2020).
S. A. Trip, Imaging in multiple sclerosis. Journal of Neurology, Neurosurgery & Psychiatry 76, iii11-iii18 (2005).
A. MacKay, et al., Insights into brain microstructure from the T2 distribution. Magnetic Resonance Imaging 24, 515-525 (2006).
K. H. Cha, et al., Evaluation of data augmentation via synthetic images for improved breast mass detection on mammograms using deep learning. Journal of Medical Imaging 7,1 (2019).
J. P. Mugler, J. R. Brookeman, Three-dimensional magnetization-prepared rapid gradient-echo imaging (3D MP RAGE). Magnetic Resonance in Medicine 15,152-157 (1990).
D. A. Feinberg, et al., Multiple spin-echo magnetic resonance imaging. Radiology 155,437-442 (1985).
J. V. Hajnal, et al., Use of Fluid Attenuated Inversion Recovery (FLAIR) Pulse Sequences in MRI of the Brain. Journal of Computer Assisted Tomography 16,841-844 (1992).
Z. Lesjak, et al., A Novel Public MR Image Dataset of Multiple Sclerosis Patients With Lesion Segmentations Based on Multi-rater Consensus. Neuroinformatics 16,51-63 (2018).
B. Fischl, et al., Whole Brain Segmentation. Neuron 33,341-355 (2002).
D. N. Greve, B. Fischl, Accurate and robust brain image alignment using boundary-based registration. Neurolmage 48,63-72 (2009).
N. W. DAW, Why After-Images are not Seen in Normal Circumstances. Nature 196,1143-1145 (1962).
S. Mehta, et al., Y-Net: Joint Segmentation and Classification for Diagnosis of Breast Biopsy Images in International Conference on Medical Image Computing and Computer-Assisted Intervention, (2018), pp. 893-901.
M. Tan, Q. Le, EfficientNet: Rethinking Model Scaling for Convolutional Neural Networks in Proceedings of the 36th International Conference on Machine Learning, Proceedings of Machine Learning Research, K. Chaudhuri, R. Salakhutdinov, Eds. (PMLR, 2019), pp. 6105-6114.
D. R. Cox, The Regression Analysis of Binary Sequences. Journal of the Royal Statistical Society: Series B (Methodological) 20,215-232 (1958).
M. L. McHugh, Interrater reliability: the kappa statistic. Biochemia Medica 22,276-282 (2012).
M. Szumilas, Explaining odds ratios. Journal of the Canadian Academy of Child and Adolescent Psychiatry 19,227-9 (2010).

What is claimed is:

1. A method of synthesizing a magnetic resonance (MR) image, the method comprising:
obtaining a quantitative MRI (qMRI) map of values of an MRI parameter;
modulating values of said MRI parameter within a region of said qMRI map, to mimic a tissue pathology therein, thereby providing a modulated qMRI map; and
generating an MR image based on said modulated qMRI map, thereby synthesizing the MR image.

2. The method according to claim 1, further comprising generating said qMRI map.

3. The method according to claim 2, wherein said generating said qMRI map is based on an MR signal acquired from a subject.

4. The method according to claim 3, wherein said subject is a healthy subject.

5. The method according to claim 1, further comprising accessing a computer readable medium storing a database having a plurality of entries each associating a database pathology to a database value or range of values of at least one MRI parameter, and searching said database for an entry having a database pathology matching said tissue pathology, wherein said modulating said values of said parameter is based on a database value or range of values of said found entry.

6. The method according to claim 1, further comprising randomly selecting said region.

7. The method according to claim 1, wherein said modulating is along a randomly selected pattern within said region.

8. The method according to claim 1, wherein said region is predetermined.

9. The method according to claim 1, further comprising accessing a computer readable medium storing a database having a plurality of entries each associating a database pathology to a database morphology, and searching said database for an entry having a database pathology matching said tissue pathology, wherein said modulating said values within said region is along a pattern selected based on a database morphology of said found entry.

10. The method according to claim 1, comprising receiving input pertaining to a severity level of said tissue pathology, wherein said modulating said values is based on said received severity level.

11. The method according to claim 1, comprising generating a simultaneous graphical output of said synthesized the MR image, and an MR image corresponding to said qMRI map prior to said modulation.

12. A computer software product, comprising a computer-readable medium in which program instructions are stored, which instructions, when read by a data processor, cause the data processor to receive a qMRI map of values of at least one MRI parameter and execute the method according to claim 1.

13. A method of training an artificial neural network, comprising:
executing a method of synthesizing a magnetic resonance (MR) image a plurality of times to respectively synthesize a plurality of MR images, each associated with at least one tissue pathology;
feeding the artificial neural network with said synthesized MR images and said respective tissue pathologies, to obtain weight parameters for the artificial neural network; and
storing the weight parameters in a computer readable medium;
wherein said method of synthesizing an MR image is the method of claim 1.

14. The method according to claim 13, further comprising re-executing said method of synthesizing an MR image an additional plurality of times to respectively synthesize an additional plurality of MR images, each associated with at least one tissue pathology;
validating said weight parameters by feeding the artificial neural network with each of said additional plurality of synthesized MR images, and comparing an output of the artificial neural network with a respective tissue pathology; and
generating a report indicative of said validation.

15. A computer software product, comprising a computer-readable medium in which program instructions are stored, which instructions, when read by a data processor, cause the data processor to receive a qMRI map of values of at least one MRI parameter and execute the method according to claim 13.

16. A computer software product for training a user, the computer software product comprising a computer-readable medium in which program instructions are stored, which instructions, when read by a data processor, cause the data processor to:
display on a display device a graphical user interface (GUI) having a training activation control;
automatically execute the method according to claim 1, responsively to an activation of said control by the user; and
generate a graphical output of said synthesized the MR image on said GUI.

17. The computer software product according the claim 16, wherein said program instructions, when read by a data processor, cause the data processor to synthesize an ordered set of MR images mimicking said tissue pathology, and to generate a graphical output separately for each of said MR images on said GUI.

18. The computer software product according to claim 17, wherein said set of MR images comprises synthesized MR images at which a visibility of said synthesized pathology gradually increases or decreases.

19. The computer software product according to claim 18, wherein said set of MR images comprises synthesized MR images at which a severity level of said synthesized pathology gradually increases or decreases.

20. The computer software product according to claim 18, wherein said set of MR images comprises synthesized MR images at which a size of said region gradually increases or decreases.
